(19) United States
(12) Patent Application Publication
(10) Pub. No.: US 2026/0000510 A1
(43) Pub. Date: Jan. 1, 2026

(54) Heart Valve Delivery Device

(71) Applicant: Magnolia Medical Corp., Newport Beach, CA (US)

(72) Inventors: Christian G. MONROE, San Diego, CA (US); Ari W. ROGERS, West Orange, NJ (US); Jack Berkman SATTELL, Cambridge, MA (US)

(73) Assignee: Magnolia Medical Corp., Newport Beach, CA (US)

(21) Appl. No.: 19/132,288

(22) PCT Filed: Nov. 28, 2023
(86) PCT No.: PCT/US2023/081439

§ 371 Date: May 22, 2025

Related U.S. Application Data
(60) Provisional application No. 63/385,185, filed on Nov. 28, 2022.

(51) Int. Cl. A61F 2/24

(52) U.S. Cl. CPC A61F 2/2436

(57) ABSTRACT

A delivery system for a heart prosthesis includes a tubular loading member with a notch passing through its wall at a distal edge, and a prosthesis retention member positioned within the tubular loading member. The prosthesis retention member has multiple prosthesis retention areas shaped to engage prosthesis engagement portions of a heart prosthesis. The tubular loading member and the prosthesis retention member are longitudinally and rotationally movable relative to each other. The notch has multiple rotational positions that completely expose one of the prosthesis retention areas while covering the remaining areas. This delivery system allows for precise positioning and secure retention of the heart prosthesis during implantation.

### RELATED APPLICATIONS

This application claims benefit of and priority to U.S. Provisional Application Ser. No. 63/385, 185 filed Nov. 28, 2022 entitled Valve Delivery System, which is hereby incorporated herein by reference in its entirety.

### BACKGROUND

Heart valve disease is a common condition affecting millions of people worldwide. The heart has four valves, which regulate blood flow by opening and closing during each heartbeat. When these valves become damaged or diseased, they may not function properly, leading to a variety of symptoms such as shortness of breath, fatigue, and chest pain. In severe cases, heart valve disease can lead to heart failure or even death.

Traditional treatment for heart valve disease involves surgical replacement of the affected valve with a prosthetic valve. While this procedure is effective, it is invasive and requires a significant recovery period. Additionally, some patients may not be eligible for surgery due to other health conditions.

In recent years, there has been growing interest in minimally invasive techniques for heart valve replacement, such as Transcatheter Aortic Valve Replacement (TAVR). This technique involves inserting a collapsible valve into the heart through a catheter, typically inserted into the femoral artery. The valve is then deployed within the damaged valve, replacing it and restoring normal blood flow.

More recently, there has been a growing interest in using this technique for the replacement of the mitral and tricuspid valves, known as Transcatheter Mitral Valve Replacement (TMVR) and Transcatheter Tricuspid Valve Replacement (TTVR), respectively. These valves are more complex than the aortic valve, and their replacement using traditional surgical techniques can be challenging. TMVR and TTVR offer a less invasive option for patients with mitral or tricuspid valve disease, who may not be eligible for traditional surgical valve replacement.

TMVR and TTVR require specialized devices, which are designed to fit within the unique shape of the mitral or tricuspid valve. These devices are typically made of biocompatible materials and are designed to be deployed through a catheter, similar to the TAVR procedure.

Overall, TMVR and TTVR offer a promising new option for patients with mitral or tricuspid valve disease, who may not be eligible for traditional surgical valve replacement. As with any new medical technology, there are still many challenges to be addressed, including device design, patient selection, and long-term outcomes. However, the potential benefits of these techniques make them an exciting area of research and development in the field of cardiology.

### SUMMARY

In some aspects, the techniques described herein relate to a delivery system for a heart prosthesis, including: a tubular loading member including a notch passing entirely through a wall of the tubular loading member and located at a distal edge of the tubular loading member; and, a prosthesis retention member positioned within the tubular loading member; the prosthesis retention member including a plurality of prosthesis retention areas shaped to engage prosthesis engagement portions of a heart prosthesis; wherein the tubular loading member and the prosthesis retention member are longitudinally and rotationally movable relative to each other; and, wherein the notch includes a plurality of rotational positions that completely radially expose one of the plurality of prosthesis retention areas while covering a remaining portion of the plurality of prosthesis retention areas.

In some aspects, the techniques described herein relate to a delivery system, wherein the plurality of prosthesis retention areas are positioned circumferentially around a body of the prosthesis retention member.

In some aspects, the techniques described herein relate to a delivery system, wherein the notch is about a same size or larger than each of the plurality of prosthesis retention areas.

In some aspects, the techniques described herein relate to a delivery system, wherein the notch has a width and shape sized to allow the prosthesis engagement portions pass through the notch.

In some aspects, the techniques described herein relate to a delivery system, wherein the tubular loading member includes a prosthesis loading tool having a tubular structure; wherein the tubular loading member is removable from the delivery system.

In some aspects, the techniques described herein relate to a delivery system, wherein the tubular loading member includes a distal region having a reduced diameter relative to a proximal region.

In some aspects, the techniques described herein relate to a delivery system, further including a funnel member removably connected to the tubular loading member.

In some aspects, the techniques described herein relate to a delivery system, further including a notch cover member that is removably positioned over the notch of the tubular loading member and at least partially around the tubular loading member.

In some aspects, the techniques described herein relate to a delivery system, wherein the notch cover member further includes a ridge positioned on an inner surface of the notch cover member and that is sized to fit within the notch of the prosthesis loading tool.

In some aspects, the techniques described herein relate to a delivery system, wherein the notch cover member is an open tubular shape.

In some aspects, the techniques described herein relate to a delivery system, wherein the prosthesis engagement portions are enlarged regions on a proximal portion of the heart prosthesis.

In some aspects, the techniques described herein relate to a delivery system, wherein the enlarged regions are eyelets.

In some aspects, the techniques described herein relate to a delivery system, wherein the plurality of prosthesis retention areas include depressions having rectangular shape adjacent to a circular shape.

In some aspects, the techniques described herein relate to a delivery system, wherein the plurality of prosthesis retention areas include depressions, grooves, posts, walls, or any combination of the same.

In some aspects, the techniques described herein relate to a delivery system wherein the tubular loading member includes a first tubular member and the notch is located at a distal edge of the first tubular member; wherein the first tubular member is non-removably fixed to the delivery system.

In some aspects, the techniques described herein relate to a delivery system, further including a first tubular member longitudinally movable relative to the prosthesis retention member.

In some aspects, the techniques described herein relate to a delivery system, further including a second tubular member positioned within a lumen of the first tubular member and connected to the prosthesis retention member.

In some aspects, the techniques described herein relate to a delivery system, further including a shaft positioned within a lumen of the second tubular member; wherein a distal region of the shaft includes a nosecone.

In some aspects, the techniques described herein relate to a delivery system, further including a third tubular member disposed around the first tubular member, wherein the third tubular member forms an outer tube of the delivery system.

In some aspects, the techniques described herein relate to a delivery system, further including a handle assembly, wherein the handle assembly includes a movement mechanism connected to the first tubular member that moves the first tubular member distally and proximally relative to the handle assembly.

In some aspects, the techniques described herein relate to a delivery system, wherein the movement mechanism includes an outer tube that rotates relative to an outer handle housing; and wherein an inner surface of the outer tube is engaged with a nut via threaded surfaces that cause the nut to longitudinally move when the outer tube is rotated; and wherein the nut is connected to the first tubular member.

In some aspects, the techniques described herein relate to a delivery system, wherein the first outer tube includes a distal tubular portion included of a rigid material and includes a proximal tubular portion included of a flexible material.

In some aspects, the techniques described herein relate to a delivery system, wherein the heart prosthesis is a heart valve including an atrial flange portion, a leaflet engaging portion, and a body portion.

In some aspects, the techniques described herein relate to a delivery system, wherein the leaflet engaging portion include a plurality of engagement struts that are positioned proximally of the body portion when the heart valve is in a radially compressed configuration and positioned alongside the body portion when the heart valve is in a radially expand configuration.

In some aspects, the techniques described herein relate to a method of loading a heart prosthesis on to a delivery system, including: positioning a proximal end of the heart prosthesis near a prosthesis retention member of a delivery device; rotating a tubular loading member at a distal region of the delivery device so that a notch on a distal end of the tubular loading member aligns with a first prosthesis retention area of plurality of prosthesis retention areas; placing a first prosthesis engagement portion of a heart prosthesis through the notch and into first prosthesis retention area of the plurality of prosthesis retention areas; rotating the tubular loading member so that the notch aligns with a second prosthesis retention area of the plurality of prosthesis retention areas; placing a second enlarged retention portion of the heart prosthesis through the notch and into the second prosthesis retention area of the plurality of prosthesis retention areas; further rotating the tubular loading member, aligning the notch with further retention area of the plurality of prosthesis retention areas, and placing further enlarged retention portions of the heart prosthesis into corresponding retention areas of the plurality of prosthesis retention areas; and, covering the notch to prevent the heart prosthesis from radially expanding.

In some aspects, the techniques described herein relate to a method, wherein the tubular loading member is a prosthesis loading tool that is removable from the delivery device or a first tubular member that is non-removably fixed to the delivery device.

In some aspects, the techniques described herein relate to a method, wherein covering the heart prosthesis includes placing notch cover member 134 over the notch.

In some aspects, the techniques described herein relate to a method, wherein placing a spacer member over the notch further includes placing a ridge on an inner surface of the spacer into the notch.

In some aspects, the techniques described herein relate to a method, wherein covering the heart prosthesis includes retracting the prosthesis retention member and the heart prosthesis into first tubular member of the delivery device.

In some aspects, the techniques described herein relate to a method, further including removing the prosthesis loading tool and the notch cover member.

In some aspects, the techniques described herein relate to a method, positioning the proximal end of the heart prosthesis through a funnel member located at a distal end of the prosthesis loading tool.

In some aspects, the techniques described herein relate to a method, wherein retracting the prosthesis retention member and the heart prosthesis into the first tubular member of the delivery device further includes rotating a first portion of a handle connected to a proximal end of the first tubular member.

### BRIEF DESCRIPTION OF THE DRAWINGS

The following figures are included to illustrate certain example aspects of the present disclosure and should not be viewed as exclusive or limiting. The subject matter disclosed is capable of considerable modifications, alterations, combinations, and equivalents in form and function, as will occur to one having ordinary skill in the art and having the benefit of this disclosure. The present disclosure references the drawings as follows:

FIG. 1 is a side view of a prosthesis delivery device.

FIG. 2 is a side view of the prosthesis delivery device of claim 1.

FIG. 3 is a magnified view of area 3 of FIG. 1.

FIG. 4 is a magnified view of area 4 of FIG. 2.

FIG. 5 is a magnified view of area 5 of FIG. 2.

FIG. 6 is a magnified view of area 6 of FIG. 2.

FIGS. 7A and 7B are views of a prosthesis retention member.

FIGS. 8A and 8B are views of a funnel member.

FIGS. 9A and 9B are views of a notch cover member.

FIGS. 10A and 10B are views of a prosthesis loading tool.

FIG. 11 is a view of a distal end of the delivery device of FIG. 1.

FIG. 12 is a view of a distal end of the delivery device of FIG. 1.

FIG. 13 is a view of a distal end of the delivery device of FIG. 1.

FIG. 14 is a view of a distal end of the delivery device of FIG. 1.

FIG. 15 is a view of a distal end of the delivery device of FIG. 1.

FIG. 16 is a view of a distal end of the delivery device of FIG. 1.

FIG. 17 is a view of a distal end of the delivery device of FIG. 1.

FIG. 18 is a view of a distal end of a delivery device for a prosthesis.

FIG. 19 is a perspective view of a prosthetic heart valve.

FIG. 20 is a view of the prosthetic heart valve of FIG. 19.

FIG. 21 is a side view of the prosthetic heart valve of FIG. 19.

FIG. 22 is a bottom view of the prosthetic heart valve of FIG. 19.

FIG. 23 is a top view of the prosthetic heart valve of FIG. 19.

FIG. 24 is a cross-sectional view of the prosthetic heart valve of FIG. 19.

FIG. 25 is a cross-sectional view of the prosthetic heart valve of FIG. 19.

FIG. 26 is an enlarged view of the prosthetic heart valve of FIG. 19.

FIG. 27 is an enlarged view of the prosthetic heart valve of FIG. 19.

FIG. 28 is an enlarged view of the prosthetic heart valve of FIG. 19.

FIG. 29 is an enlarged view of the prosthetic heart valve of FIG. 19.

FIG. 30 is a perspective view of a framework of the prosthetic heart valve of FIG. 19.

FIG. 31 is a side view of a framework of the prosthetic heart valve of FIG. 19.

FIG. 32 is a side view of a framework of the prosthetic heart valve of FIG. 19.

FIG. 33 is a top view of a framework of the prosthetic heart valve of FIG. 19.

FIG. 34 is a bottom view of a framework of the prosthetic heart valve of FIG. 19.

FIG. 35 is an enlarged view of a framework of the prosthetic heart valve of FIG. 19.

FIG. 36 is an enlarged view of a framework of the prosthetic heart valve of FIG. 19.

FIG. 37 is an enlarged view of a framework of the prosthetic heart valve of FIG. 19.

FIG. 38 is an enlarged view of a framework of the prosthetic heart valve of FIG. 19.

FIG. 39 is an enlarged view of a framework of the prosthetic heart valve of FIG. 19.

FIG. 40 is a cross-sectional view of a framework of the prosthetic heart valve of FIG. 19.

FIG. 41 is a simplified line view of several components of the prosthetic heart valve of FIG. 19.

FIG. 42 is a view of the prosthetic heart valve of FIG. 19 with valve leaflets.

FIG. 43 is a side view of the prosthetic heart valve of FIG. 19 in a native heart valve.

FIG. 44 is a side view of the prosthetic heart valve of FIG. 19 in a native heart valve.

FIG. 45 is a side view of the prosthetic heart valve of FIG. 19 in a native heart valve.

### DETAILED DESCRIPTION

It will be appreciated by persons skilled in the art that the present disclosure is not limited to what has been particularly shown and described herein. A variety of modifications and variations are possible in view of the teachings herein without departing their scope, spirit, or intent.

While different examples may be described in this specification, it is specifically contemplated that any of the features from the different examples can be used and brought together in any combination. In other words, the features of different examples can be mixed and matched with each other. Hence, while every permutation of features from different examples may not be explicitly shown or described, it is the intention of this disclosure to cover any such combinations, especially as may be appreciated by one of skill in the art.

The terminology used in this disclosure should be interpreted in a permissive manner and is not intended to be limiting. In the drawings, like numbers refer to like elements. Unless otherwise noted, all of the accompanying drawings are not to scale. Unless otherwise noted, the term “about” is defined to mean plus-or-minus 5% of a stated value.

The terms distal or distally generally refer to a direction or area towards an end of a device within a patient (e.g., away from a physician/clinician), while the terms proximal or proximally refer to a direction or area toward an end of a device that remains outside of a patient (e.g., toward or closer to a physician/clinician or handle/hub of a device).

The present disclosure is generally directed to delivery devices for heart prostheses. For example, heart prostheses may include an artificial heart valve, a heart valve remodeling device such as an annuloplasty ring, or similar devices that are delivered within a valve of a heart. In the example of artificial heart valves, these may be used to replace any of the native human heart valves (e.g., aortic valve, mitral valve, pulmonary valve, or tricuspid valve), but may be particularly useful for replacing a mitral or tricuspid valve. Additionally, the delivery device of this disclosure may be used for devices intended for use in areas other than a heart, such as a vascular stent.

Generally, the present artificial heart valves described in this disclosure include a support structure that supports artificial valve leaflets or similar mechanisms that allow blood to generally flow in only one direction through the artificial heart valve. It should be understood that the use of leaflets and similar structures with the support structures are specifically contemplated. In other words, while some of this disclosure may describe aspects of artificial valve support structures, the artificial valve as a whole is specifically included as part of the present examples.

When referring to the artificial valves and support structures in this specification, the terms “top end,” “inflow end,” and similar variants may be used interchangeably to mean an end of the device through which blood normally first enters the valve/device. For example, referring to a tricuspid valve, the end of the device in or closes to the right atrium. The terms “bottom end,” “outflow end,” and similar variants may be used interchangeably to mean an end of the device through which blood normally exits the valve/device. For example, referring to a tricuspid valve, the end of the device in or closest to the right ventricle. In addition, the artificial valves and support structures in this specification may be referred to as having proximal ends/portions and distal ends/portions in the context of a delivery device/catheter. Typically, the term proximal indicates a portion or direction along the delivery device closer to the physician and distal indicates a portion or direction along or away from the delivery device in the opposite direction of the physician. In some of the examples described in this specification, the top or inflow end of the support structure may also be the proximal end, and the bottom or outflow end of the support structure may also be the distal end.

Similarly, with regard to discussion of the delivery system or device, the terms distal or distally generally refer to a direction or area towards an end of the device within a patient (e.g., away from a physician/clinician), while the terms proximal or proximally refer to a direction or area toward an end of the device that remains outside of a patient (e.g., toward or closer to a physician/clinician or handle/hub of a device).

The present disclosure is generally directed to a delivery system for a heart prosthesis, such as an artificial heart valve. The present disclosure is also generally directed to a method of loading a delivery system with a heart prosthesis and a method of delivering a heart prosthesis. The delivery system may allow a heart prosthesis to be more easily loaded into the delivery system prior to a procedure and may allow for the heart prosthesis to be better positioned and deployed within a patient's heart, as discussed in further detail below.

The delivery system may comprise a first tube and a prosthesis retention member located within a lumen of the first tube and near a distal region of the first tube. The prosthesis retention member may comprise a retention body with a plurality of prosthesis retention areas that portions of a heart prosthesis may be positioned into or onto. The prosthesis retention member may be movable relative to the first tube such that when the prosthesis retention member is positioned within the first tube, it prevents the release of the heart prosthesis (e.g., via radial expansion).

The retention body of the prosthesis retention member may be solid, solid with a passage therethrough, or tubular in shape. The plurality of prosthesis retention areas may be located around a diameter perpendicular to a longitudinal axis of the delivery device. The plurality of prosthesis retention areas may include depressions or recesses, as well as raised surfaces such as walls, posts, tabs, longitudinally oriented slots, or any combination of the prior, as well as similar structures. The plurality of prosthesis retention areas may be spaced at equal distances from each other or at non-equal distances from each other. In one specific example, the plurality of prosthesis retention areas are recesses with a mating shape of distal engagement members of the heart prosthesis to fit within. In another specific example, the prosthesis retention areas are recesses with a post that fits within an eyelet of an engagement member of the heart prosthesis. The engagement members of the heart prosthesis may include areas of enlarged width, areas of enlarged height, areas with an aperture such as an eyelet, curved/bent regions such as hooks or wavy areas, or any combination of these features.

The heart prosthesis may be loaded onto or into the delivery device via a notch that can be rotationally positioned over one of plurality of prosthesis retention areas of the prosthesis retention member while the remaining prosthesis retention areas remain covered (note, “notch” may include slots, openings, passages, or similar structures). This may be achieved via a removable prosthesis loading tool or via structures built into the delivery device itself.

In one example, a prosthesis loading tool may comprise a generally tubular shape with a notch located along a distal edge of the tubular shape. The notch may extend entirely through a wall of the tubular shape, creating an opening or passage from a position radially outside of the tubular shape to within an interior lumen of the tubular shape. The notch may have a width that is large enough to allow engagement members of the heart prosthesis to pass through. The notch may have a generally uniform width or may increase and/or decrease in width between its proximal and distal ends to create various shapes (e.g., distally increasing width, distally decreasing width, distally increasing and then decreasing width, distally decreasing and increasing width, circular shapes, triangular shapes, rectangular shapes, square shapes, rectangular and circular shapes combined, or any combination of these shapes and variations thereof). The prosthesis loading tool may rotate around the prosthesis retention member to allow each of the engagement members of the heart prosthesis to be engaged with the prosthesis retention areas.

In another example, a first tube of the delivery device may include the notch as described above. The first tube may move longitudinally relative to the prosthesis retention member and may also rotate relative to the prosthesis retention member (or a distal portion of the first tube may rotate relative to a proximal portion of the first tube). In this respect, the first tube of the delivery device may act in a similar manner to the prosthesis loading tool, but otherwise be integrated into the delivery device itself. In one example, the outer tube is longitudinally movable proximally and distally relative to the prosthesis retention member, such as via longitudinal hand movements or via a movement mechanism in a proximal handle of the delivery device. Alternatively, the prosthesis retention member may be movable relative to the first tube such as via longitudinal hand movements or via a movement mechanism in a proximal handle of the delivery device.

The prosthesis loading tool or notched first tube may further comprise a removable engagement member that may be placed over the notch after the heart prosthesis is fully positioned on the prosthesis retention member to prevent the engagement member of the heart prosthesis from radially expanding out of engagement with a prosthesis retention area of the prosthesis retention member. The removable engagement member may include a tube or “C” shaped clip sized to be positioned over the notch, and may further include a ridge or raised surface on an inside surface of the removable engagement member that fits within the notch.

The prosthesis loading tool or notched first tube may further comprise a funnel member that may be positioned over a distal portion of the prosthesis loading tool or the notched first tube to help allow the heart prosthesis to be proximally withdrawn into the delivery device after its engagement members have been engaged with the plurality of prosthesis retention members.

The delivery device may further comprise a nosecone that is longitudinally moveable in proximal and distal directions relative to the prosthesis retention member and the first tube. In a loading configuration, the nosecone may be distally spaced apart from the first tube and the prosthesis retention member. After a heart prosthesis is retained by the prosthesis retention member and covered by the first tubular member, the nosecone may move to a delivery configuration in which it abuts or is directly adjacent to the distal end of the first tube and the distal end of the heart prosthesis. The nose cone may be connected and move via an elongated shaft or tube that extends to a proximal end of the delivery device.

The delivery device may further comprise a second tube that forms an outer tube of the delivery device, being positioned around and over at least most of the first tube. In one example, the second tube is connected to a proximal handle and fixed from longitudinal movement relative to the handle, the first tube is connected to the proximal handle and is longitudinally movable with a movement mechanism of the handle and relative to the handle, and the shaft connected to the nose cone is connected to the handle and is longitudinally movable relative to the handle.

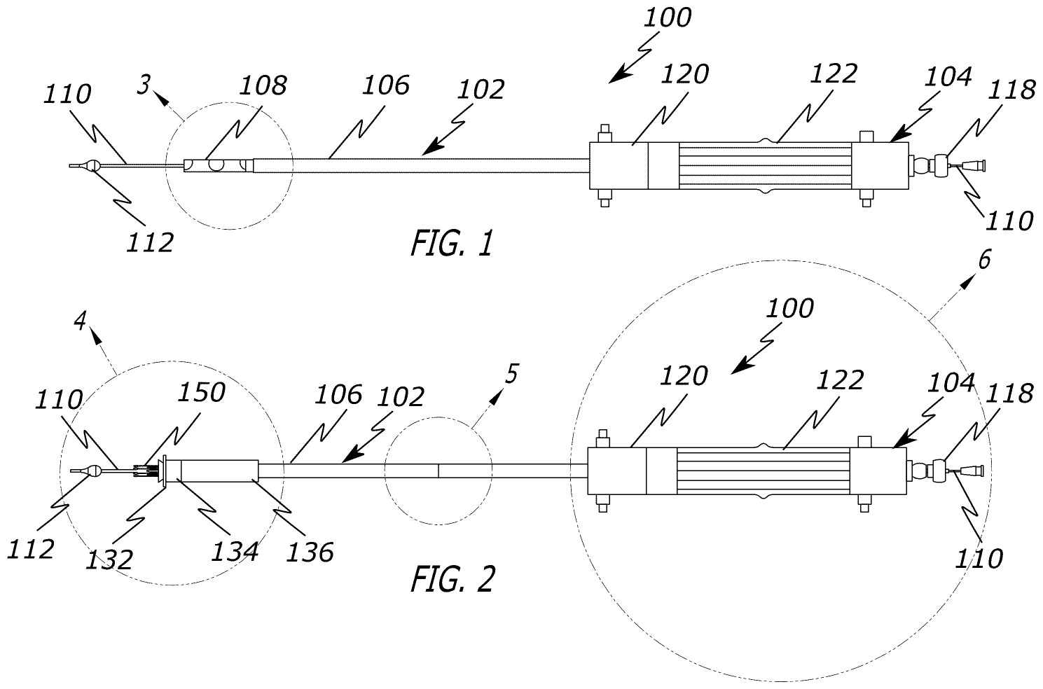

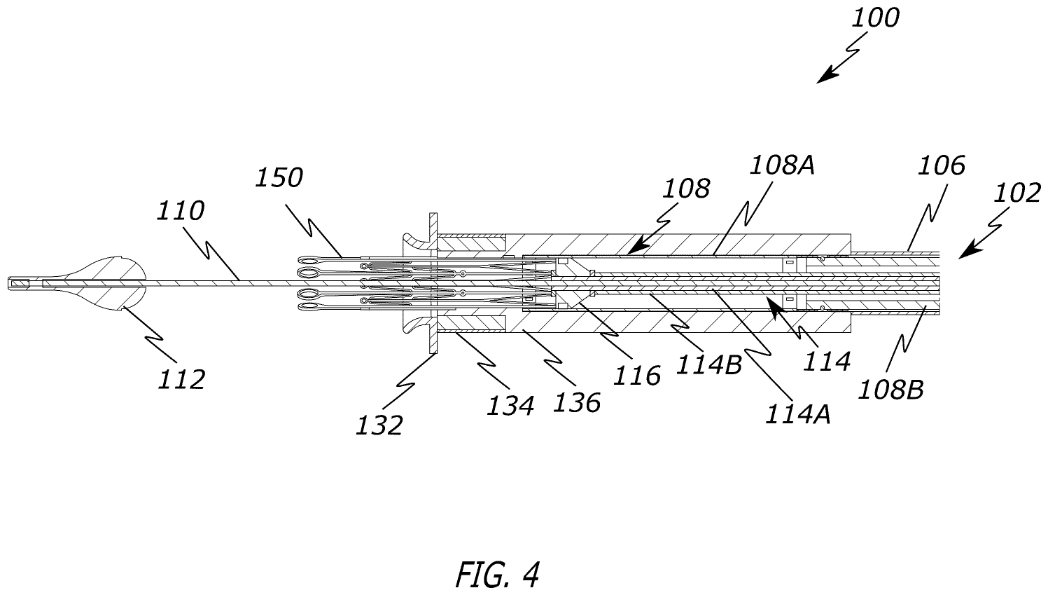

FIGS. 1-18 illustrate various aspects of example delivery systems 100 and delivery device 100′ for a heart prosthesis. In these figures, the heart prosthesis is depicted and described as an artificial heart valve 150. However, other types of heart prosthesis are possible, such as heart annuloplasty rings, stents, or similar devices. As discussed in greater detail below, the artificial heart valve 150 is engaged with a prosthesis retention member 116 that can be loaded via a prosthesis loading tool 136 with a notch 162A or distal tubular portion 108A with a notch 108C that allows engagement members on the artificial heart valve 150, such as eyelets 162A. Generally, either the prosthesis loading tool 136 with distal region 136A or distal tubular portion 108A with notch 108C may be generally referred to a tubular loading member, since they both have generally tubular shapes and may be used for loading the artificial heart valve 150.

Turning first the example of FIGS. 1-17, FIG. 1 illustrates a side view of the delivery device 100. Generally, the delivery device 100 has an elongated body assembly 102 that has a length sufficient to reach a target location within a human body, such as a human heart valve. As described further below, the artificial heart valve 150 may be loaded into and deployed out of the elongated body assembly 102.

The delivery device 100 may also include a handle assembly 104 that may control longitudinal movement of one or more components of the elongated body assembly 102 and that is connected to a proximal end of the handle assembly.

FIG. 2 illustrates a side view of the delivery device 100 during a loading procedure of the artificial heart valve 150 into the elongated body assembly 102. In the present example, a prosthesis loading tool 136, a notch cover member 134, and a funnel member 132 are all removably located on or near a distal portion or end of the elongated body assembly 102 and used to load the artificial heart valve 150, as described further below.

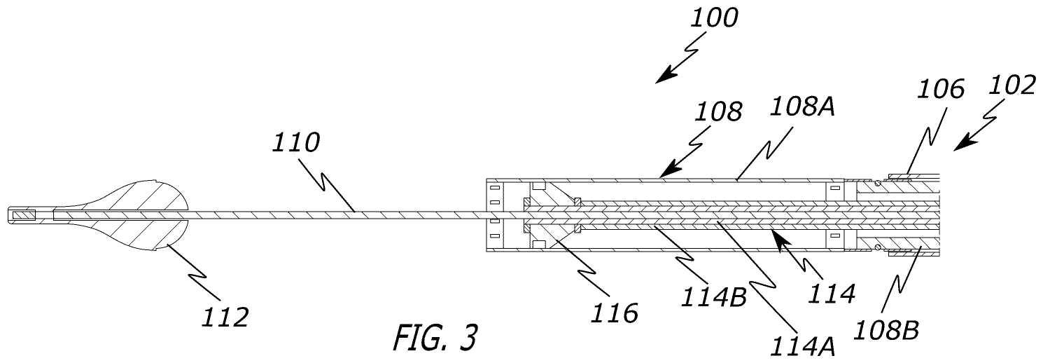

FIG. 3 illustrates a cross sectional view of a distal portion (Area 3 from FIG. 1) of the elongated body assembly 102. The distal portion may comprise a prosthesis retention member 116 onto which the artificial heart valve 150 is engaged with a first tubular member 108. In the present example, the first tubular member 108 may be moved longitudinally while the prosthesis retention member 116 remains in place relative to the handle assembly 104. However, the prosthesis retention member 116 may alternatively be configured to move longitudinally while the first tubular member 108 remains in place relative to the handle assembly 104.

In the present example, the first tubular member 108 may be composed of a distal tubular portion 108A that is connected to a distal end of a proximal tubular portion 108B. The distal tubular portion 108A may be composed of a more rigid material, such as a biocompatible metal (e.g., stainless steel, Nitinol, a rigid polymer, etc.) and may have a length that covers all of or at least a portion of the artificial heart valve 150 when retracted into the distal tubular portion 108A. The proximal tubular portion 108B may be composed of a relatively flexible polymer (e.g., solid or braided polyurethane) which may allow its length to have enough flexibility to pass through a tortuous path within the body, such as into a human heart. Hence, the first tubular member 108 may have both rigid properties to help maintain the artificial heart valve 150 in a compressed state and flexible properties to help the elongated body assembly 102 to pass through the vasculature of a patient.

Alternatively, instead of two discrete, attached sections, the first tubular member 108 may be a single, unitary tubular structure. The unitary tubular structure may have a uniform and constant construction throughout its length or may vary its construction along its length to achieve different flexibilities. In one example, the first tubular member 108 may be composed of a polymer tube (e.g., polyurethane) and may incorporate reinforcements at one or more locations. Braided wires, coiled wires, or similar structures may be attached or embedded in the polymer tube, such as along a distal region of the first tubular member 108 such that it covers all or a portion of the artificial heart valve 150 when fully retracted into the first tubular member 108.

The prosthesis retention member 116 may be connected to the handle assembly 104 via either a tube or a shaft. In the present example, a second tubular member 114 is connected to the prosthesis retention member 116 and to the handle assembly 104. The second tubular member 114 may include an inner tubular layer 114A that connected to a distal end of the prosthesis retention member 116 and an outer tubular layer 114B that connects to a proximal end of the prosthesis retention member 116 (or vice versa), however the second tubular member 114 may alternatively be a single tubular layer that connects to either the distal or proximal end of the prosthesis retention member 116.

The delivery device 100 may include a nosecone 112 that may be positioned distally of the prosthesis retention member 116 and first tubular member 108, for example via a shaft 110. The shaft 110 may be moved longitudinally from a proximal end of the delivery device 100 to move the nosecone 112 distally or proximally. The nosecone 112 may help create a gradual or tapered distal end of the delivery device 100 and may further help retain the artificial heart valve 150 within the first tubular member 108. The nosecone 112 may be comprised of a variety of different materials such as braided polyimide or other polymers. The nosecone 112 may have a shape that increases in diameter from its distal end. The nosecone 112 may also have a shape that decreases along only a proximal portion so as to create an overall bulbus shape, as seen in FIG. 3.

The elongated body assembly 102 may also include a third tubular member 106 that forms an outer tube or layer to the elongated body assembly 102 and which the first tubular member 108, second tubular member 114, and shaft 110 are at least partially positioned within. In the present example, the third tubular member 106 is connected to the handle assembly 104 and extends to a location proximal of the distal tubular portion 108A when the first tubular member 108 is extended over the prosthesis retention member 116. This positioning allows the first tubular member 108 to be retracted proximally during use to expose and deploy the artificial heart valve 150.

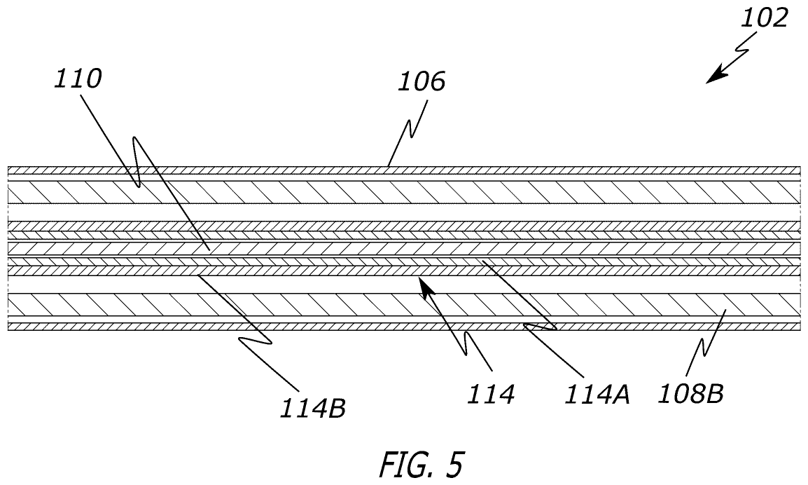

FIG. 5 is a cross sectional view of Area 5 from FIG. 2 showing a middle region of the elongated body assembly 102, including the third tubular member 106 forming an outer tubular layer, the proximal tubular portion 108B forming an inner tubular layer, the second tubular member 114 forming an inner tubular layer within proximal tubular portion 108B, and the shaft 110 located within second tubular member 114.

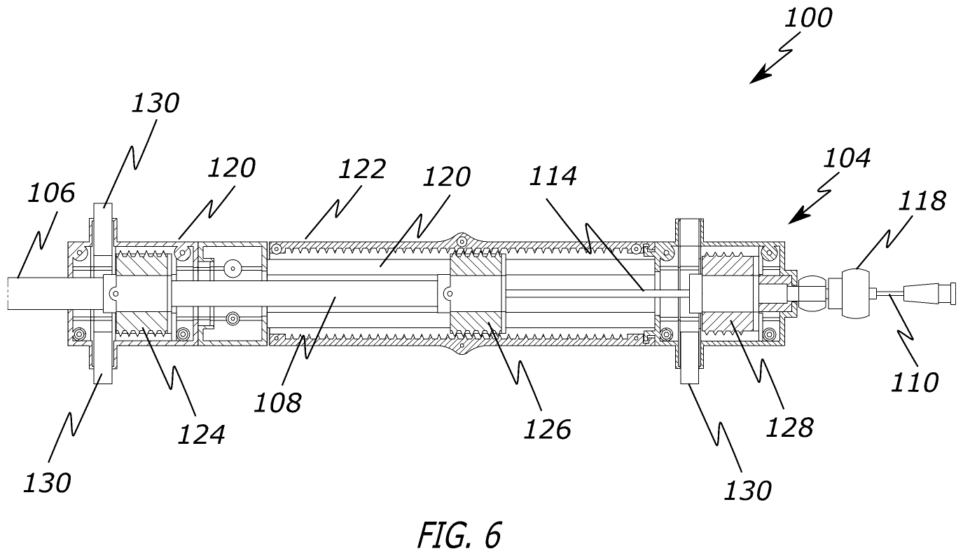

FIG. 6 illustrates a cross sectional view of the handle assembly 104 of Area 6 in FIG. 2. The handle assembly 104 may include an outer handle housing 120 that at least partially encompasses the third tubular member 106, the proximal tubular portion 108B, second tubular member 114, and shaft 110.

As previously discussed, the first tubular member 108 may be moved longitudinally in a proximal and distal direction relative to the elongated body assembly 102 to load the artificial heart valve 150 and release the artificial heart valve 150 during delivery to a target location (e.g., a heart valve). The first tubular member 108 may be moved via a movement mechanism in the elongated body assembly 102. In one example, the movement mechanism comprises an outer tube 122 (e.g., a tubular knob, tubular dial, or tubular thumb wheel) that may have an inner threaded surface. A first nut member 126 is positioned within a lumen of the outer tube 122 and has an outer threaded surface that mates with an engages the inner threaded surface of the outer tube 122. The nut member 126 is also fixed to the first tubular member 108. In that respect, as the outer tube 122 is rotated by the user, it causes the first nut member 126 to move either proximally or distally within the handle assembly 104, thereby also moving the first tubular member 108 relative to the handle assembly 104 and other components of the delivery device 100. Again, in other examples, this first nut member 126 may instead be connected to the second tubular member 114 to longitudinally move the prosthesis retention member 116 relative to the first tubular member 108.

The third tubular member 106 that forms an outer layer of the elongated body assembly 102 may be fixed to the outer handle housing 120. For example, the third tubular member 106 may be connected to a second nut member 124 that is maintained in place relative to the outer handle housing 120, thereby maintaining the third tubular member 106 in the same position relative to the outer handle housing 120. In another example, the third tubular member 106 may be directly connected to the outer handle housing 120 (e.g., via adhesives).

The second tubular member 114 that connects to the prosthesis retention member 116 may also be fixed to the outer handle housing 120. For example, the second tubular member 114 may be connected to a third nut 128 that is maintained in place relative to the outer handle housing 120, thereby maintaining the second tubular member 114 in the same position relative to the third tubular member 106. In another example, the second tubular member 114 may be directly connected to the outer handle housing 120 (e.g., via adhesives).

The shaft 110 that connects to the nosecone 112 may also be supported by the handle assembly 104. In one example, the shaft 110 extends through the outer handle housing 120 and through a clamping valve 118, such as a touhy-borst valve that has a nut that when tightened, clamps or otherwise closes its passage. In this manner, the clamping valve 118 can be tightened or loosened to prevent or allow movement of the shaft 110 respectively.

The handle assembly 104 may also include one or a plurality of luer fittings 130 (e.g., 1, 2, 3, 4) that provide fluid connections into the handle assembly 104 and/or the various tubes of the elongated body assembly 102.

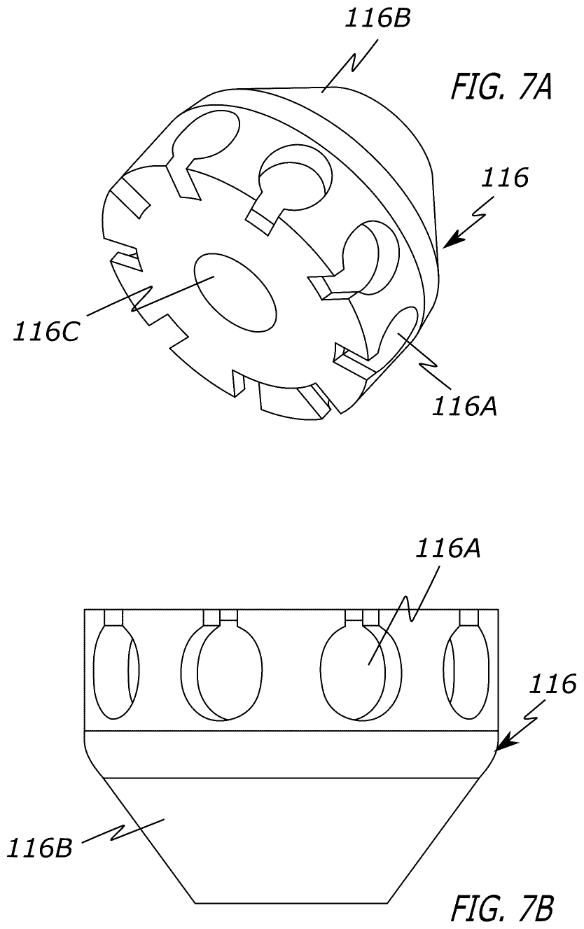

FIGS. 7A and 7B illustrate magnified views of the prosthesis retention member 116 that is connected to the distal portion of the second tubular member 114. The prosthesis retention member 116 may comprise a body member that at least partially comprises a cylindrical shape. The cylindrical shape may have a plurality of prosthesis retention areas 116A that are positioned around the outer surface of the cylindrical portion. The plurality of prosthesis retention areas 116A may comprise shapes that engage prosthesis engagement portions 162A of the artificial heart valve 150. For example, the plurality of prosthesis retention areas 116A may include depressions, slots, apertures, slots, walls, ridges, posts, shafts or similar shapes that, once engaged, prevent the prosthesis engagement portions 162A from longitudinally disengaging from the prosthesis retention member 116 but allow radial expansion of the artificial heart valve 150 to disengage the prosthesis engagement portions 162A from the plurality of prosthesis retention areas 116A.

The prosthesis retention member 116 may also comprise a proximal conical surface 116B and a passage 116C extending axially through the prosthesis retention member 116 that the second tubular member 114 may pass through, as well as the shaft 110.

In the present example, the prosthesis engagement portions 162A may be regions of the artificial heart valve 150 near its proximal end that increase in width to form a generally circular shape. This generally circular shape may include an aperture or eyelet. The plurality of prosthesis retention areas 116A may have a similar mating shape with a distal narrow region and a wider, rounded proximal portion. In this respect, one of the prosthesis engagement portions 162A may be radially compressed into one of the 116A, and when then prevented from radial expansion, that engaged one of the prosthesis engagement portions 162A is prevented from longitudinal movement relative to the prosthesis retention member 116.

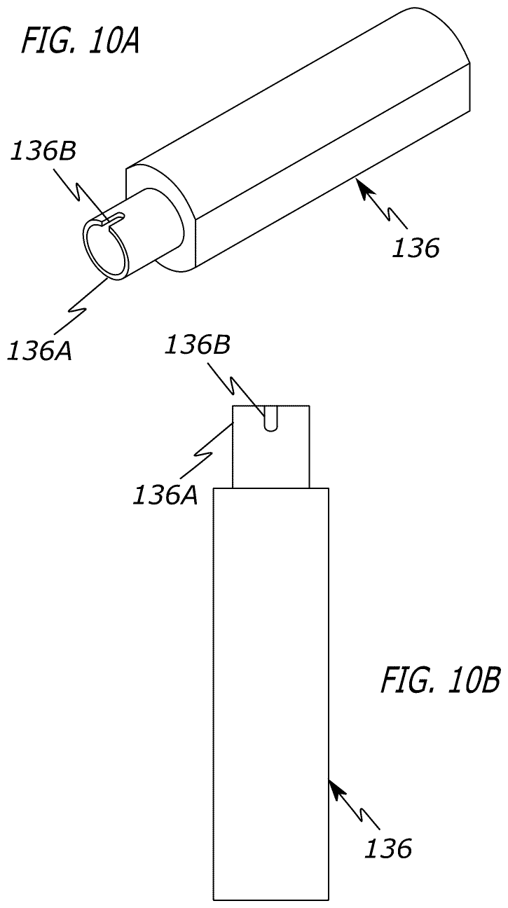

As previously discussed, there are several different possible mechanisms and/or techniques for loading the artificial heart valve 150 on to the prosthesis retention member 116. The first is via a prosthesis loading tool 136 shown best in FIGS. 2, 10A and 10B. The prosthesis loading tool 136 may comprise a tubular structure with a distal region 136A that includes a notch 136B. The notch 136B may pass entirely through a wall of the tubular structure such that an area radially outside of the tubular structure is connected and in communication with a lumen of the tubular structure. While the term notch 136B is used, this feature may also be considered a slot, groove, opening, or aperture.

The notch 136B may have a shape that allows the prosthesis engagement portions 162A to radially pass through from outside the tubular structure to within the tubular structure where they can be positioned in the plurality of prosthesis retention areas 116A. For example, the notch 136B may have a generally rectangular shape or “U” shape with a width that is larger than the largest width of the prosthesis engagement portions 162A. In another example, the notch 136B may have a shape similar to that of the prosthesis engagement portions 162A and plurality of prosthesis retention areas 116A, such as a rectangular shape with a larger, rounded proximal shape, but that is overall larger in width to allow the prosthesis engagement portions 162A to pass through the notch 136B. The notch 136B may also extend from a distal edge of the tubular structure. In one example, the prosthesis loading tool 136 may comprise only one notch 136B. In another example, the prosthesis loading tool 136 may comprise a plurality of notches 136B, such as 2, 3, 4, 5, 6, 7, 8 or more notches 136B. Alternatively, the prosthesis loading tool 136 may have an open tubular structure, such as a “C” shape cross section with a channel extending along its entire length. In such an example, the channel may be sized to take the place of the notch 136B.

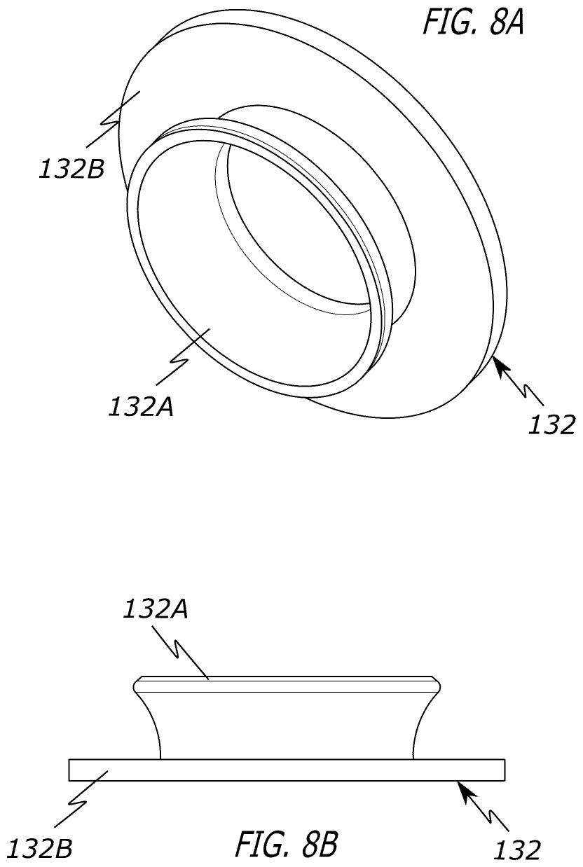

The prosthesis loading tool 136 may also have a reduced diameter region that may assist in connection of a funnel member 132 and a notch cover member 134, if needed. The funnel member 132 can best be seen in FIGS. 2, 8A, and 8B, having a funnel portion 132A that increases in diameter in a distal direction, as well as a flange portion 132B located proximally of the funnel portion 132A. The funnel member 132 may be placed on the distal region 136A of the prosthesis loading tool 136 and used to help load or funnel the artificial heart valve 150 into the delivery device 100 as described further below. In another example, the funnel member 132 may be an integrated unitary part of the prosthesis loading tool 136. In another example, the funnel member 132 may not be used during a procedure.

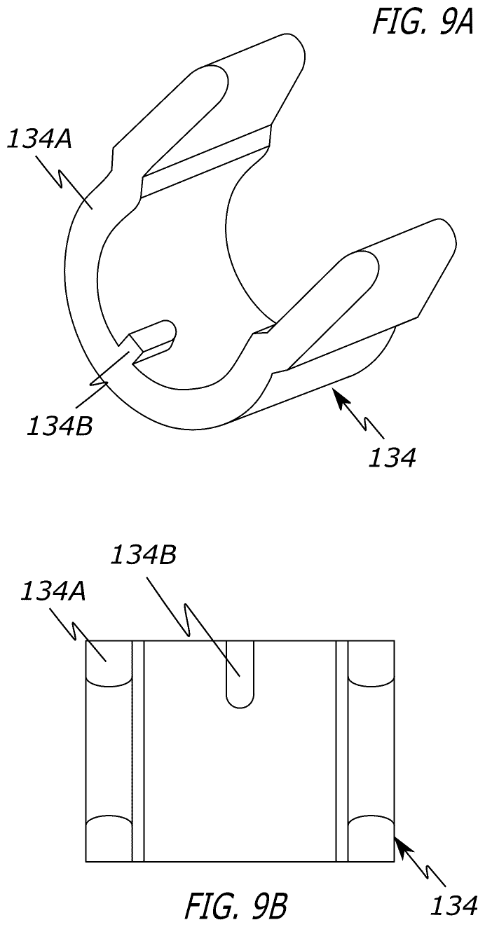

The notch cover member 134 is best seen in FIGS. 2, 9A, and 9B, and may have a body portion 134A and a ridge portion 134B. The body portion 134A may have a “C” shape that allows it to clip on to the distal region 136A of the prosthesis loading tool 136 or may alternatively have a tubular shape. The ridge portion 134B may be a raised shape from the inner surface of the body portion 134A and is sized to fit within the notch 136B of the prosthesis loading tool 136. In this respect, the notch cover member 134 can be placed over the distal region 136A after the prosthesis engagement portions 162A have been engaged with the plurality of prosthesis retention areas 116A so that the ridge portion 134B may block the notch 136B and prevent any of the prosthesis engagement portions 162A from radially moving out of the notch 136B.

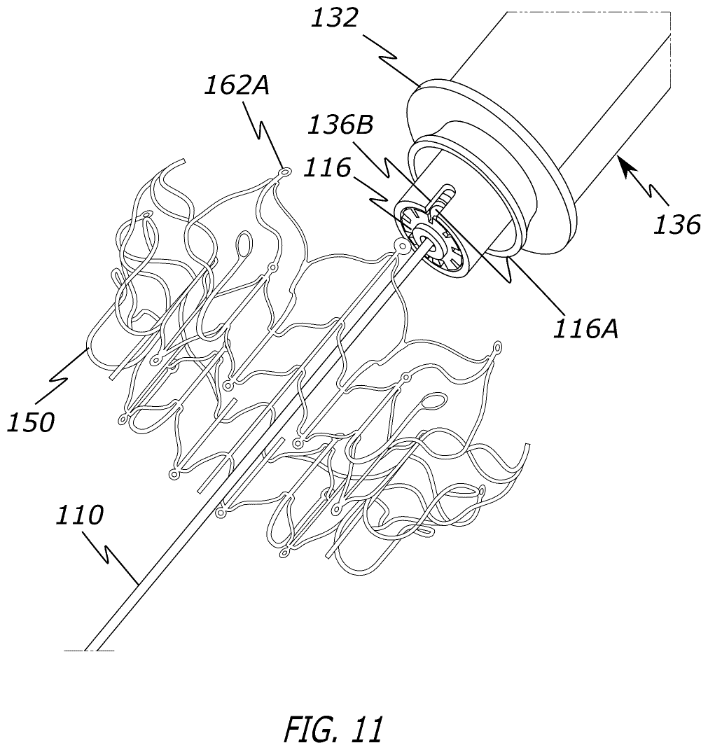

FIG. 11 illustrates an initial step in the process of loading the artificial heart valve 150 into the delivery device 100. First, the prosthesis loading tool 136 may be placed around/over the nosecone 112 and shaft 110, and moved proximally so that it may cover distal tubular portion 108A and may abut the third tubular member 106 (however, other positions of the prosthesis loading tool 136 are possible, either proximally or distally). If the funnel member 132 is being used, it may also be placed on the distal region 136A of the prosthesis loading tool 136. The distal edge of the distal region 136A of the prosthesis loading tool 136 may be positioned near the prosthesis retention member 116 so that the notch 136B may be aligned with one of the plurality of prosthesis retention areas 116A.

Next, the artificial heart valve 150 may be placed around/over the nosecone 112 and shaft 110, and moved proximally so that its prosthesis engagement portions 162A are located near the notch 136B and prosthesis retention member 116. Note that the artificial heart valve 150 is depicted only with its support structure for clarity purposes, however other components of the artificial heart valve 150 may be included, such as described later in this specification.

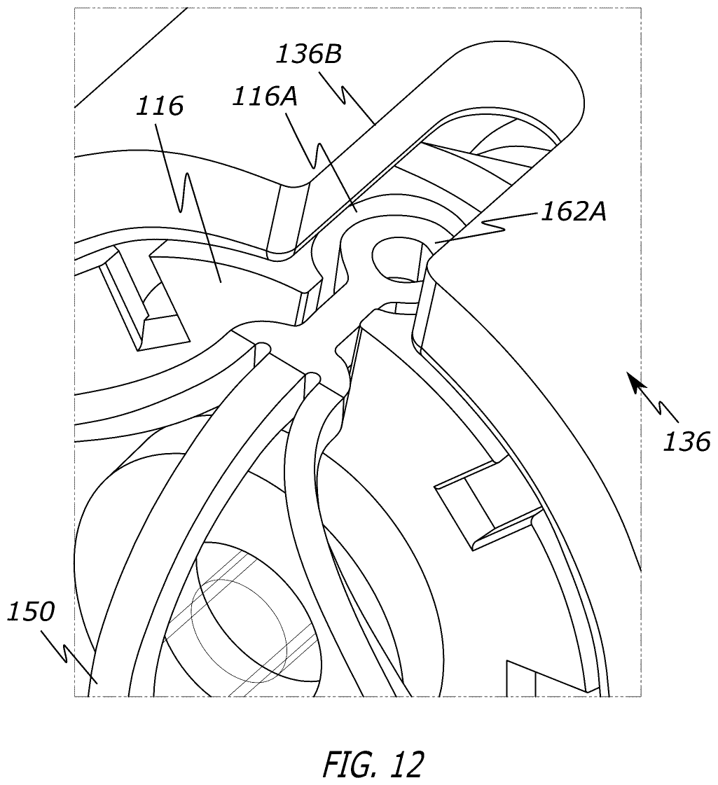

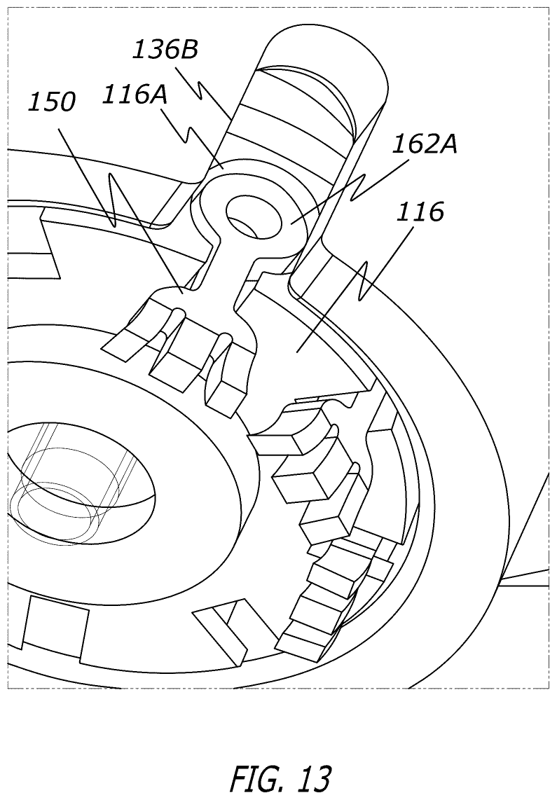

FIGS. 12 and 13 illustrate a magnified view of the notch 136B. The notch 136B is first rotationally positioned over one of the plurality of prosthesis retention areas 116A and one of the prosthesis engagement portions 162A are pushed radially through the notch 136B and into the plurality of prosthesis retention areas 116A. Next, the prosthesis loading tool 136 is rotated so that the distal region 136A is positioned over an adjacent empty plurality of prosthesis retention areas 116A and another of the prosthesis engagement portions 162A is pushed radially through the notch 136B and into the plurality of prosthesis retention areas 116A. This process is continued until all of the prosthesis engagement portions 162A are positioned in the plurality of prosthesis retention areas 116A, leaving the artificial heart valve 150 in a radially compressed configuration as seen in FIG. 14.

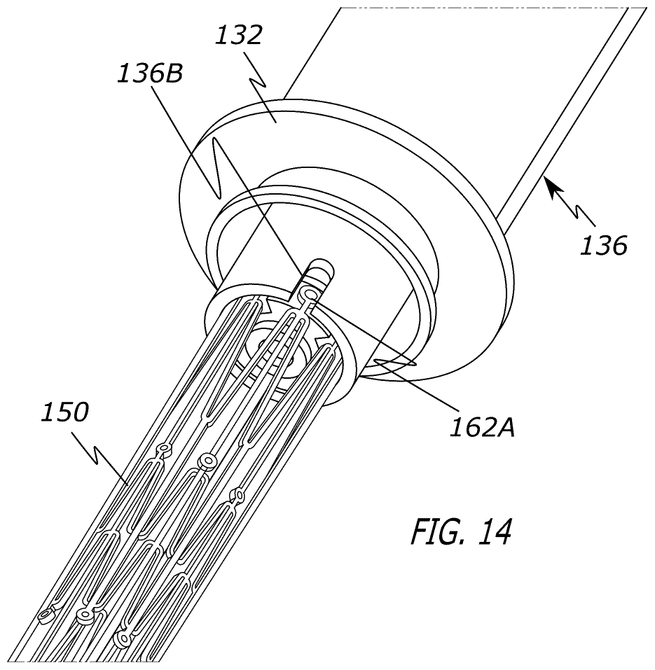

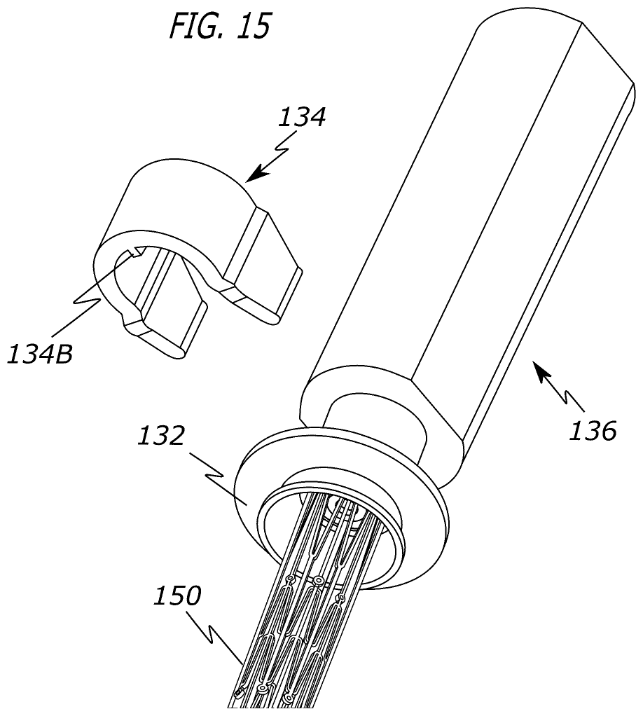

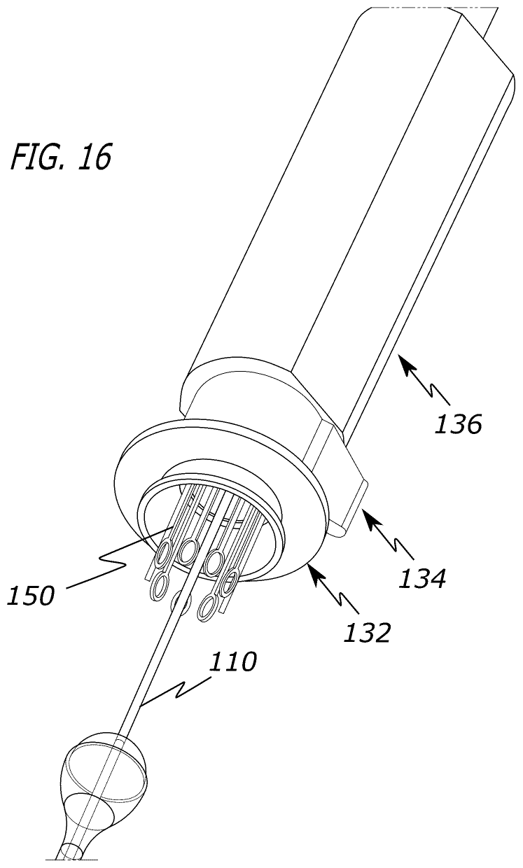

Referring to FIGS. 15 and 16, the prosthesis engagement portions 162A may be prevented from escaping the notch 136B by moving the funnel member 132 distally and placing the notch cover member 134 over the distal region 136A of the prosthesis loading tool 136 so that the ridge portion 134B blocks or fills the notch 136B.

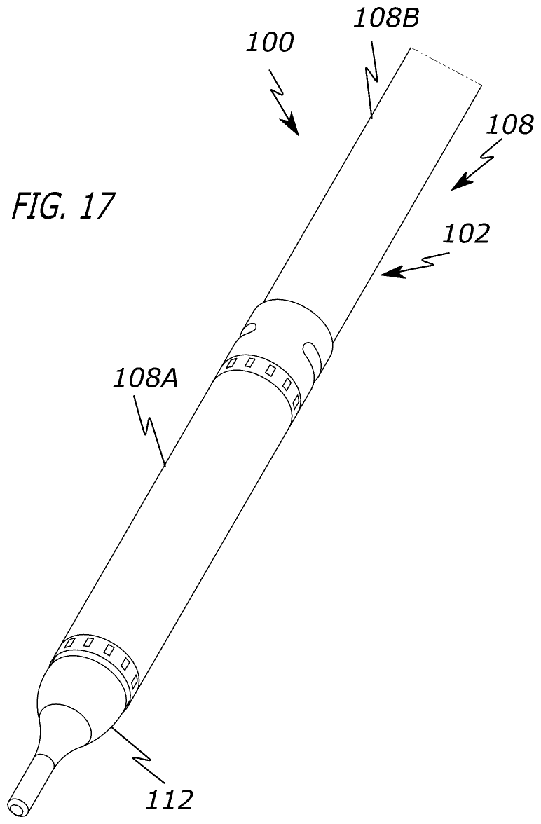

Next, the first tubular member 108 is advanced distally, such as by rotating the outer tube 122 of the handle assembly 104 until the first tubular member 108 (e.g., distal tubular portion 108A) completely covers the artificial heart valve 150. The first tubular member 108 may be further moved distally until it reaches the nosecone 112, or the shaft 110 and nosecone 112 may be moved proximally to abut the first tubular member 108 (e.g., 108A), as seen in FIG. 17. At this point, the artificial heart valve 150 may be considered loaded into the delivery device 100 and may be ready for use within a patient.

In the present example, the funnel member 132 and the notch cover member 134 are used, but the artificial heart valve 150 may also be loaded without these components. For example, after the prosthesis loading tool 136 is used to load the prosthesis engagement portions 162A into the plurality of prosthesis retention areas 116A, the notch 136B may be rotated to a position between two of the plurality of prosthesis retention areas 116A so that none of the prosthesis engagement portions 162A are released from the prosthesis loading tool 136. Next, the first tubular member 108 (e.g., distal tubular portion 108A) may be advanced distally as previously discussed to cover the artificial heart valve 150.

The prosthesis loading tool 136 is shown with a single notch 136B in the present example. However two or more notches 136B may also be included. For example, two notches 136B may be positioned at opposite locations on the distal region 136A. Alternatively, a plurality of notches 136B may be included. In one example, the distal region 136A may include a notch 136B that corresponds to every plurality of prosthesis retention areas 116A which may allow a user to load the prosthesis engagement portions 162A all simultaneously. Similarly, the notch cover member 134 may have a ridge portion 134B for each of the plurality of notches 136B.

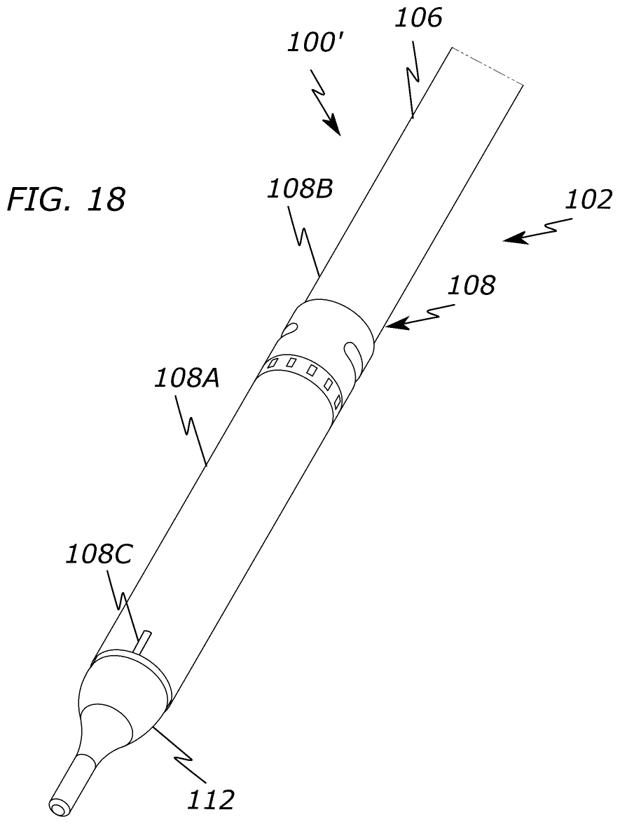

It may also be possible to load a delivery device in a similar manner with out a prosthesis loading tool 136. For example, FIG. 18 illustrates a delivery device 100′ that is otherwise the same as the previously described delivery device 100 but the distal tubular portion 108A may have its own notch 108C which is similar to the previously described notch 136B. Additionally, the distal tubular portion 108A may rotate relative to the proximal tubular portion 108B. Hence, a user may rotate the distal tubular portion 108A to place the prosthesis engagement portions 162A into the plurality of prosthesis retention areas 116A. The notch 108C may then be rotated to a position between two of the plurality of prosthesis retention areas 116A and locked rotationally in place (e.g., via a locking mechanism such as a locking pin, a longitudinal locking position, or similar locking mechanism. Again, one notch 108C may be included or a plurality of notches 108C may be included.

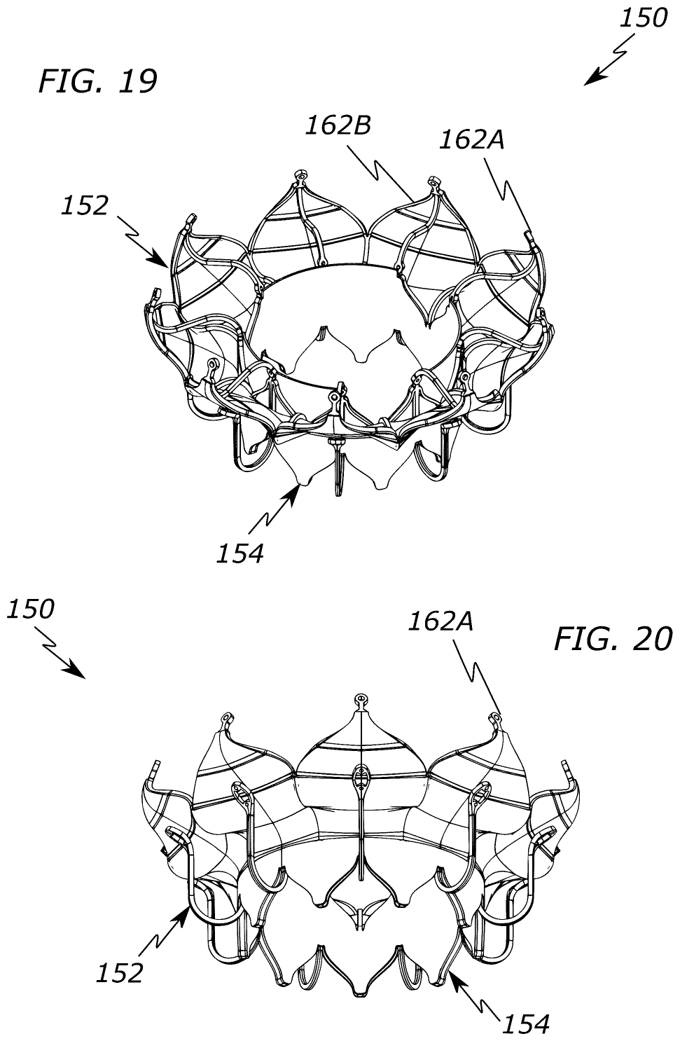

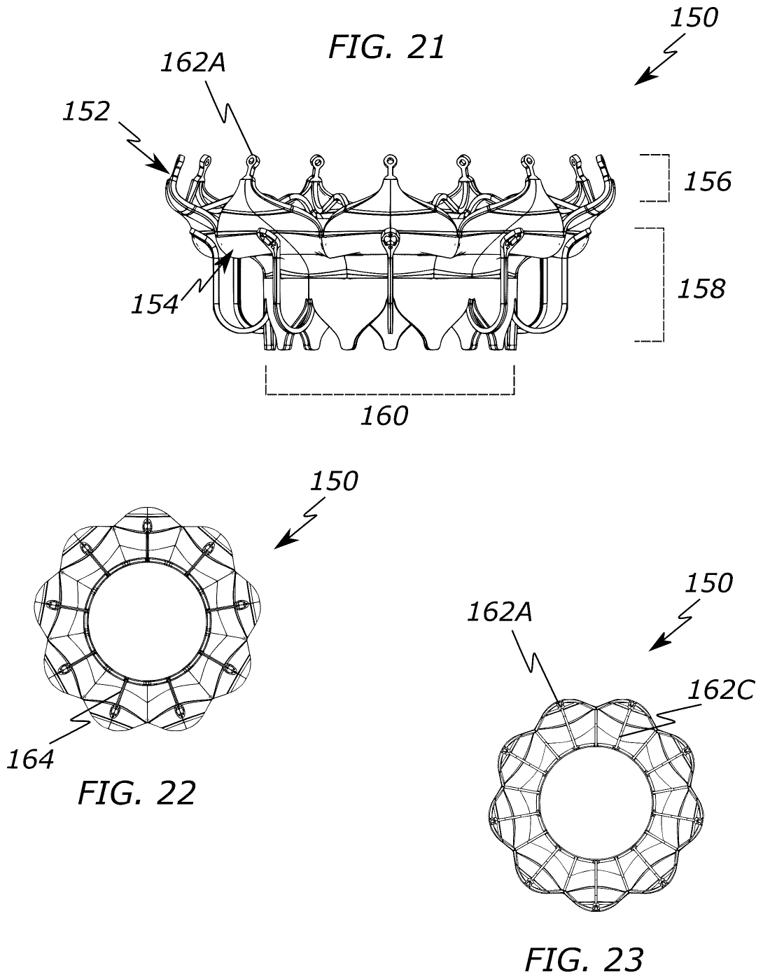

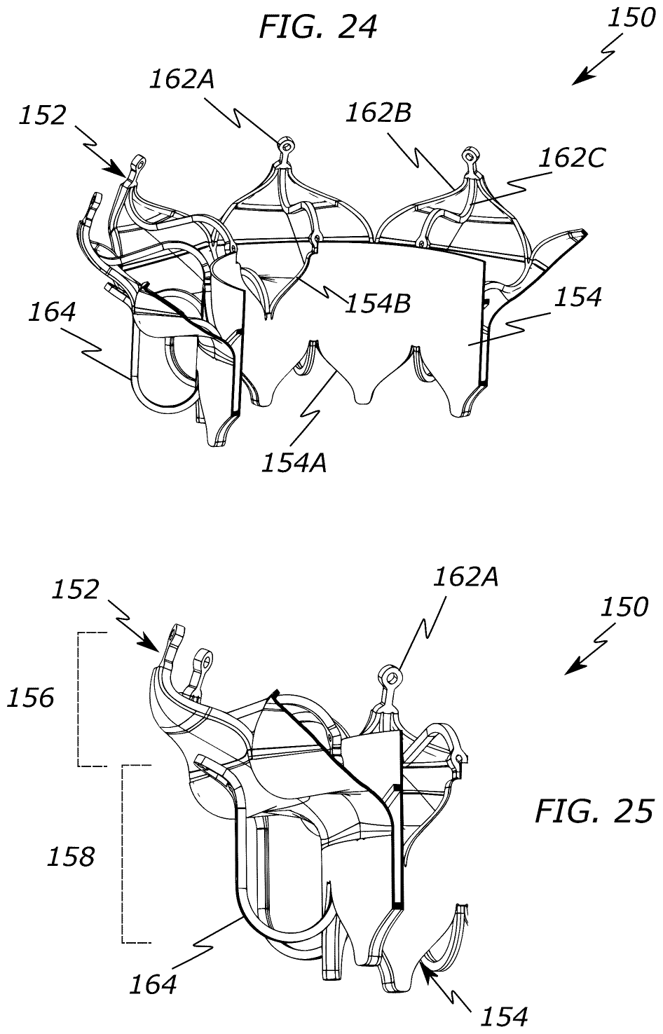

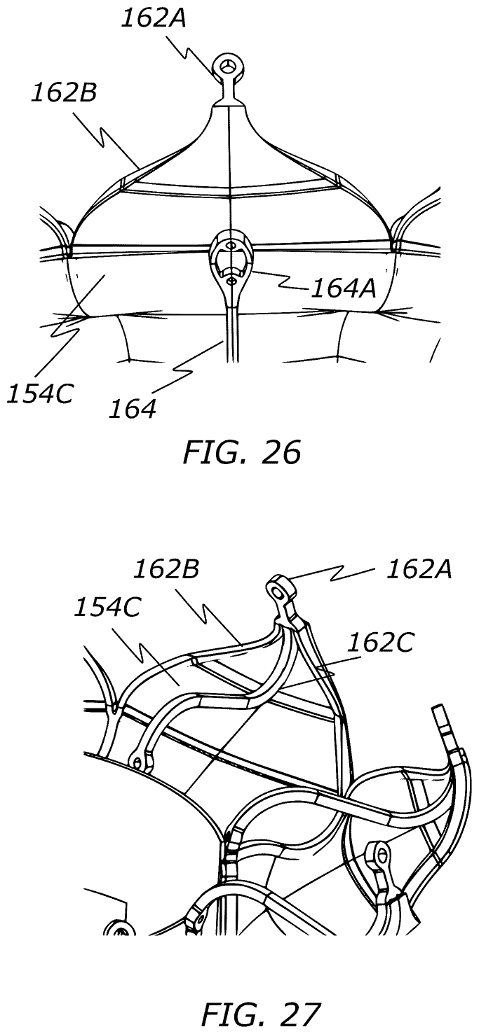

FIGS. 19-29 illustrate various aspects of an example artificial heart valve 150 that may be used with the previously described delivery device 100 or delivery device 100′. Note that some figures of this specification only show the underlying support structure of this artificial heart valve 150 and may, at times, refer to the artificial heart valve 150 as a support structure. As seen best in FIG. 19, the artificial heart valve 150 generally includes a body portion 160, an atrial flange portion 156, and a leaflet engaging portion 158.

In the present example, the body portion 160 has a generally cylindrical shape, though other shapes are possible, such as an hourglass shape, a conical shape, a concave shape, or a convex shape.

In the present example, the atrial flange portion 156 extends radially outward from a top end or an inflow end of the body portion 160. The atrial flange portion 156 may form a complete circular or annular shape beyond that of the body portion 160, though it may alternatively have other shapes such as an oval shape and may only extend around a portion of the circumference of the body portion 160 (e.g., flange regions on only opposite sides of each other). As seen best in FIG. 19, the atrial flange portion 156 also generally forms a plurality of petal shapes, pointed shapes, or outwardly narrowing shapes, such that the width of each of these areas decreases as the distance from the body portion 160 increases.

In the present example, the atrial flange 156 may have at least two regions having different angles relative to each other, as best seen in the cross-sectional view of FIG. 19. A first region initially extends radially away from the inflow end of the body portion 160. Relative to an axis extending through the inner passage of the support structure, the first region may have an angle within an inclusive range of about 70 degrees and 140 degrees (e.g., about 120 degrees). A second region radially extends from the first region and has an angle within an inclusive range of 150 degrees and 220 degrees (e.g., about 200 degrees). Generally, these two regions of the atrial flange portion 156 may help it conform to the top/inflow surface of the native valve annulus, as well walls or other areas of the atrium or leaflet/annulus.

In the present example, the leaflet engagement portion 158 may comprise a plurality of engagement struts 164 that are connected at an outflow end of the body portion 160 and extend in an inflow or upward direction. As will be described in further detail later, the engagement struts 164 curve generally parallel to the body portion 160 and then away from the body portion 160, terminating with an enlargement 164A (FIG. 20). The initial curve towards the body portion 160 may help engage or capture the native leaflets with the body portion 160. The enlargement 164A may be generally rounded to prevent damage to a patient's valve tissue and may further include one or more apertures that may be optionally used to releasably engage the artificial heart valve 150 by a delivery catheter 50.

In the present example, the engagement struts 164 are positioned at equal distances from each other around the circumference of the body portion 160. Again, non-uniform positioning of these struts 164 is also possible, such as only on opposite sides of the body portion 160 or in locations that may help avoid chordae within the ventricle.

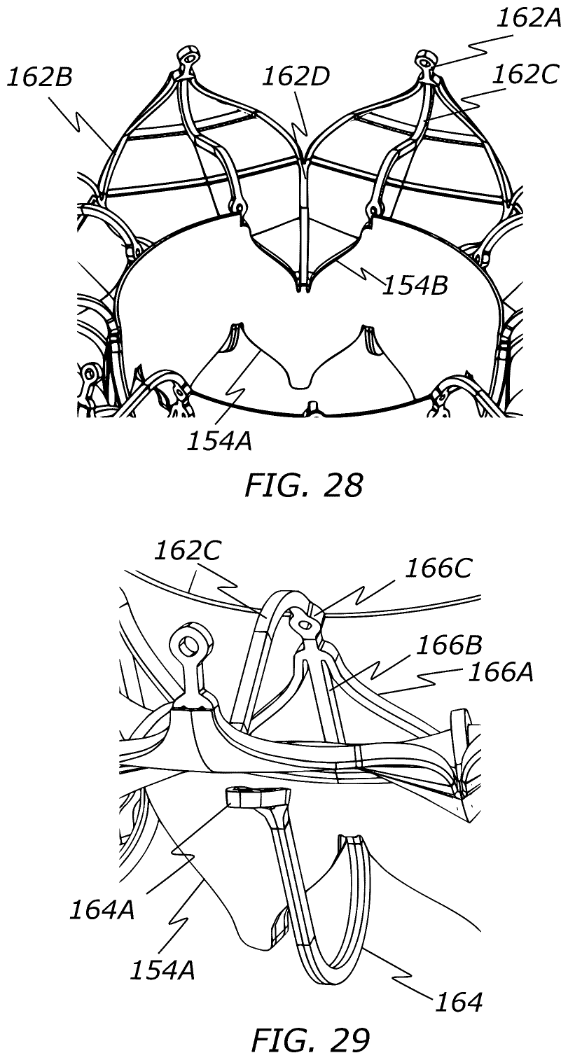

The artificial heart valve 150 in the present example includes a rigid framework 152 and a material covering 154 that is disposed over portions of the framework 152. The material covering 154 is positioned over all or most of an inside and outside of the body portion 160 of the framework 152 (seen best in FIGS. 28 and 29), and over an outside of the atrial flange portion 156 of the framework 152. The engagement struts 164 are generally left uncovered by the material covering 154. As previously described, other variations are also possible, such as locating the material covering 154 on only the inside, only the outside, and/or on any combination of the portions 156, 158, 160.

As best seen in FIGS. 28 and 29, the material covering 154 at the outflow end of the body portion 160 may form petals, pointed areas, or triangular areas 154A that generally match the underlying shapes of the framework 152 of the body portion 160. Alternatively, the outflow end of the body portion 160 may have a uniform circular-shaped edge.

Similarly, the edge of the material covering 154 at the inflow end of the body portion 160 may include one or more inset pointed or triangular gaps, spaces, or recesses 154B. While the triangular areas 154A are shown immediately adjacent to each other, the recesses 154B may be less frequent between relatively uniform edge regions. However, the inflow edge or outflow edge may take on either of the disclosed patterns in any combination, as well as have a completely uniform and perpendicular edge. As also seen in FIG. 20, the material covering 154C may also conform to the shape of the triangles or pointed petal shape of the atrial flange portion 156.

In the present example, the material covering 154 may be attached by adhesive, stitches, combinations thereof, and similar mechanisms. The material covering 154 may be composed of textile material, EPTFE sheets, PET sheets, and similar materials discussed elsewhere in this specification. In addition to the material covering 154, additional materials may be included on an underside of the atrial flange portion 156 to help create a better seal with the native valve annulus, such as hydrogel.

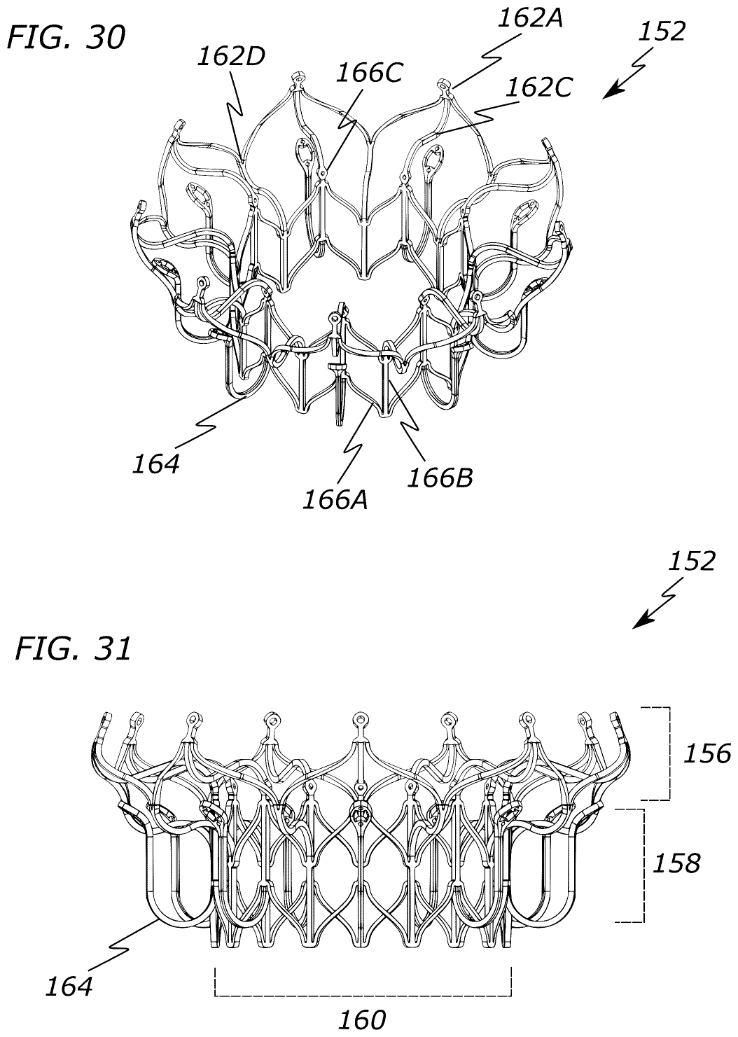

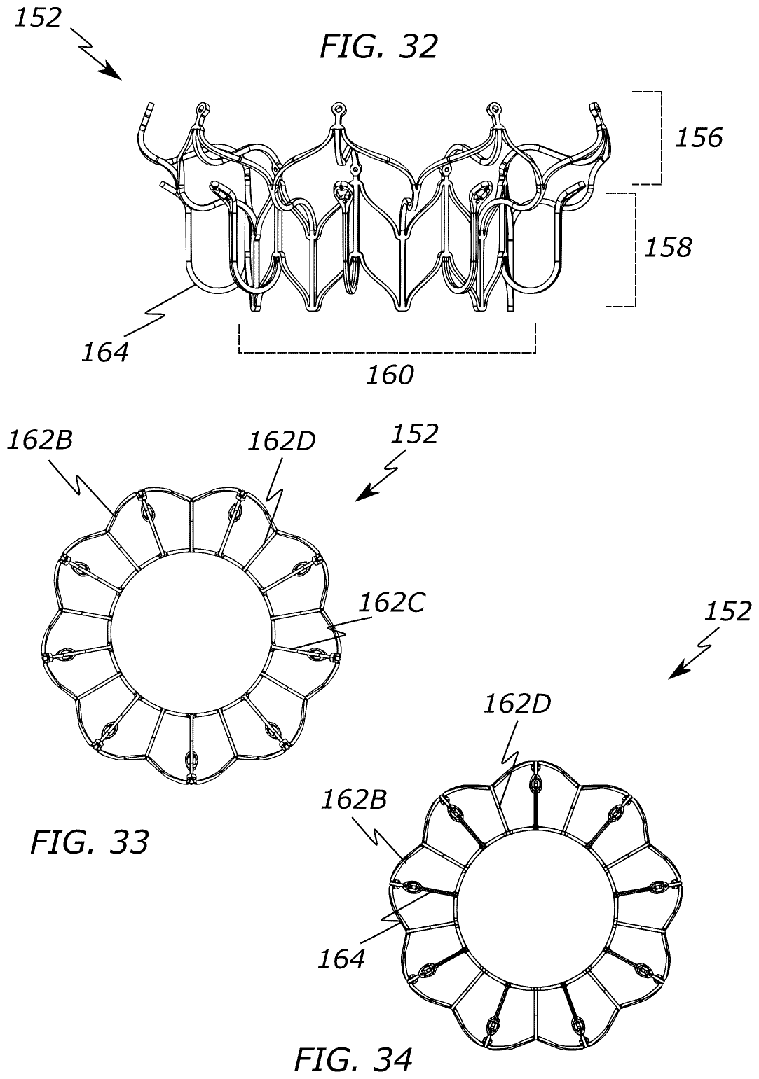

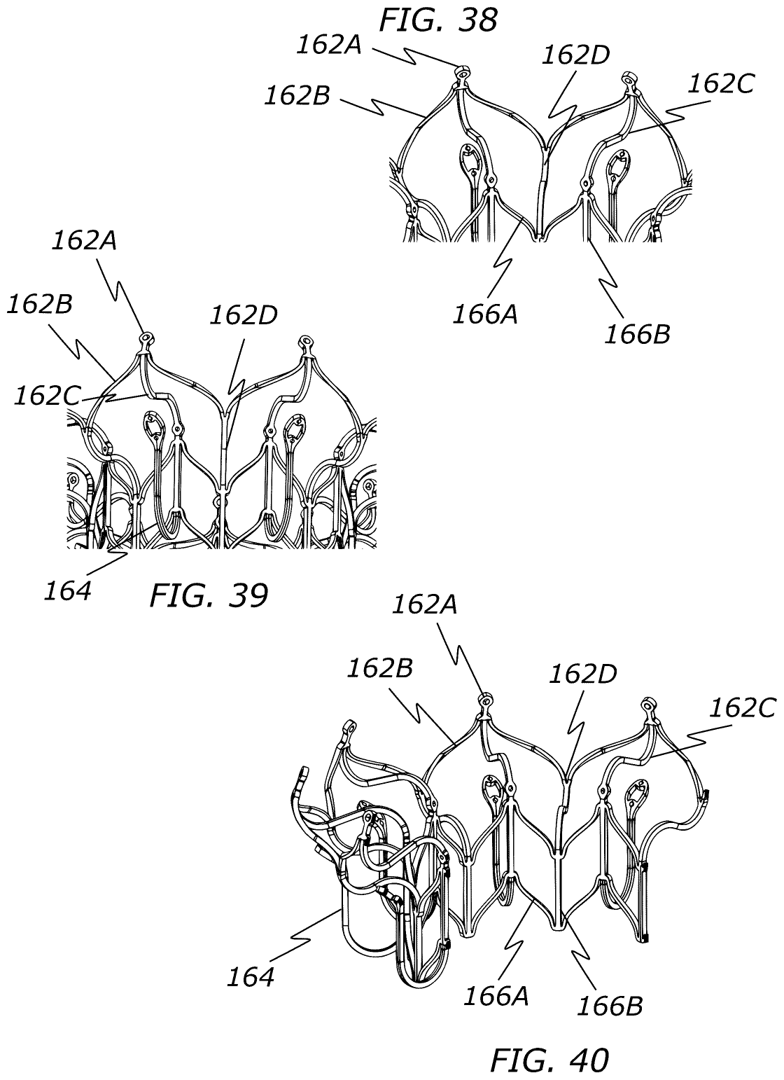

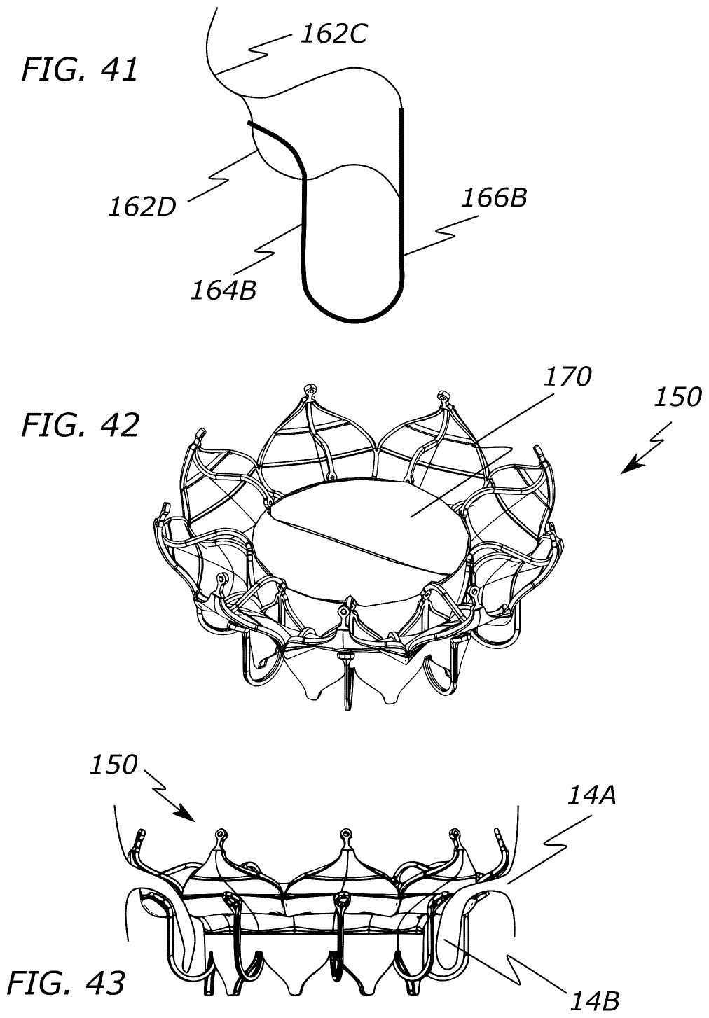

FIGS. 30-41 illustrate various aspects of the framework 152 in the present example. The body portion 160 of the framework 152 is composed of a plurality of elongated vertical body struts 166B and a plurality of horizontal body struts 166A (best seen in the cross-sectional view of FIG. 41). The vertical body struts 166B are generally parallel to an axis through the support structure's passage (i.e., an axis from the inflow end to the outflow end), while the horizontal body struts 166A are positioned around this axis in a circular shape.

The vertical body struts 166B may alternate between different heights or axial positions, such that a first vertical body strut 166B has a first axial position and the two vertical body struts 166B adjacent to the first is positioned further in an inflow direction relative to the adjacent two. Hence, the vertical body struts 166B may form an alternating pattern.

The horizontal body struts 166A may form a “V” shape or a relatively sharp angle pointing towards the outflow direction, though the opposite direction is also possible. Each end of a horizontal body strut 166A connects to a vertical body strut 166B, as well as a vertical body strut 166B passes directly through the middle of the “V” shape. The “V” shape of the horizontal body strut 166A provides a bend point at the apex of its “V” to increase and decrease its angle depending on whether the artificial heart valve 150 is in its compressed configuration or expanded configuration. In other words, the “V” shape facilitates this radial compression and expansion. Alternatively, other shapes with angles in them may also be possible for the horizontal body structure 166A, such as a “W” shape with two or more angles. In the present example, there are two rows of horizontal body struts 166A, though more rows are possible.

In one example, the body portion 160 of the framework 152 has a length within an inclusive range of about 14 mm to about 18 mm, and has a radius within an inclusive range of about 27 mm to about 30 mm.

The atrial flange portion 156 of the framework 152 includes a plurality of flange struts 162. The shape of these flange struts 162 can be best seen in FIGS. 33-41. The atrial flange portion 156 alternates with an upper radial strut 162C and a lower radial strut 162D. Both struts 162C, 162D each extend from a vertical body strut 166B. While both struts 162C, 162D may have similar shapes/size/curvature, the upper radial struts 162C are generally higher (i.e., further in an inflow direction) than lower radial struts 162D, due to the higher and lower positions of the vertical body struts 166B (e.g., due to the “V” shape/position of the horizontal body struts 166A). In the present example, an aperture portion 166C is located adjacent to the vertical body strut 166A and the upper radial strut 162. This aperture portion 166C may be optionally included for use with a delivery catheter 50.

As seen in FIG. 30, each upper radial flange strut 162C forms a first angle 162E within an inclusive range of about 90 degrees and 130 degrees, a relative straight portion 162F with a length within an inclusive range of about 3 mm and about 10 mm, a second angle 162G within an inclusive range of about 20 degrees and about 150 degrees, and a prosthesis engagement portions 162A with a length within an inclusive range of about 1 mm and 7 mm (again, angles relative to an inflow/outflow oriented axis of the artificial heart valve 150). Generally, the specific angles and sizes may vary somewhat depending on the heart and valve size of the patient. The prosthesis engagement portions 162A may optionally include an aperture that can be used by a delivery catheter 50 to help releasably retain the artificial heart valve 150 during deployment.

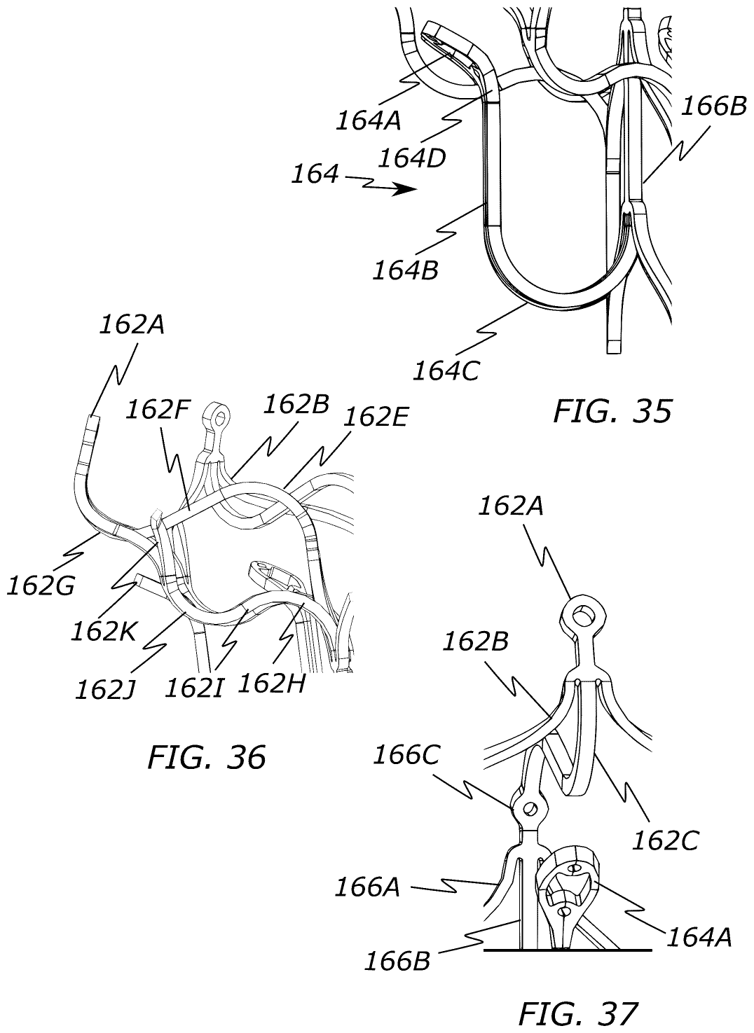

As seen in FIGS. 35 and 36, each lower radial flange strut 162D forms a first angle 162H within an inclusive range of about 90 degrees and 150 degrees, a relative straight portion 162I with a length within an inclusive range of about 0 mm and about 5 mm, a second angle 162J within an inclusive range of about 0 degrees and about 60 degrees, and a terminal portion 162K with a length within an inclusive range of about 1 mm and 7 mm (again, angles relative to an inflow/outflow oriented axis of the artificial heart valve 150). Generally, the specific angles and sizes may vary somewhat depending on the heart and valve size of the patient. The terminal portion 162K may optionally include an aperture that can be used by a delivery catheter 50 to help releasably retain the artificial heart valve 150 during deployment.

The radially outer ends of each upper radial strut 162C and lower radial strut 162D are connected to each other via one of a plurality of circumferential radial struts 162B. Since the upper radial strut 162C and lower radial strut 162D are located at different heights and distances from each other, the circumferential radial struts 162B tend to form relatively triangular or petal shapes that terminate with prosthesis engagement portions 162A. Hence, the circumferential radial struts 162B may curve in several dimensions to accommodate the upper radial strut 162C and lower radial strut 162D position difference.

The leaflet engaging portion 158 of the framework 152 includes a plurality of engagement struts 164, the shape of which can be best seen best in FIG. 35. Generally the engagement struts 164 have a straight portion 164B that is parallel to an axis through the support structure's passage (i.e., an axis from the inflow end to the outflow end). In other words, the engagement strut 164 does not angle towards the body portion 160 like the prior support structure 100, though such a configuration is possible. The engaging struts 164 are connected to the outflow end of the vertical body struts 166B. From the vertical body strut 166B, the engagement strut 164 forms a first curve 164C which curves around to about 180 degrees (e.g., an inclusive range of about 150 degrees to about 230 degrees), a first straight portion 164B with a length within an inclusive range of about 3 mm and about 10 mm, a second curve 164D curving in an opposite direction of curve 164C within an inclusive range of about 90 degrees to about 150 degrees, and a rounded portion 164A with a length within an inclusive range of about 2 mm to about 10 mm. While these curves all generally occur in the same plane, it is possible to include additional curves that may take some of the engagement struts 164 out of a single plane (i.e., curving in multiple dimensions). The rounded portion 164A may optionally include an aperture that may be used by the delivery catheter 50 to releasably retain the artificial heart valve 150 during deployment.

As previously discussed, the engagement struts 164 are not covered by the material covering 154 in the present example, but may be. Additionally, the engagement struts 164 may be coated or wrapped in a relatively softer material (e.g., a textile or EPTFE layer). Further, the rounded portion 164A of the engagement struts 164 may include a coating composed of similar materials or other materials described in this specification.

One aspect of the artificial heart valve 150 and framework 152 of note is the positions of the atrial flange 156 relative to the engagement struts 164, as seen best in the simplified line view of FIG. 41, as well as FIG. 35. In its expanded configuration, the end portions of the engagement struts 164 (i.e., portions of the leaflet engaging portion) are positioned at a more proximal location or further in an inflow direction than portions the radially-adjacent lower radial struts 162D of the atrial flange portion 156. In other words, lower radial struts 162D on each side of each of the engagement struts 164 curve axially in a distal/outflow direction beyond the end portion of the engagement struts 164. In one example, the axially-adjacent overlap is within an inclusive range of about 0.1 mm to about 10 mm.

This arrangement may be particularly helpful in several respects. First, this arrangement forces the leaflets and the annulus of the native valve to be positioned over the engagement struts 164 and then below the lower radial struts 162D, forcing the leaflets/annulus into an alternating or wave-like shape. Hence, the leaflet engaging portion 158 (i.e., engagement struts 164) and the atrial flange portion 156 (i.e., lower radial struts 162D) tend to pinch the leaflets/annulus and create a paperclip effect. This design may allow for positive remodeling (e.g., size reduction of any enlargement) of the ventricle as the body adapts to the reduced regurgitation vs the prior faulty native valve. Some other prosthetic replacement valves may be relatively large plug-like designs and may rely on radial force to anchor and seal, but the present top-down approach to sealing at the annulus may allow the ventricle to better recover over time and reduce in diameter without interference from the present replacement valve.

Second, this arrangement may hold the material covering on the underside of the atrial flange portion taut around the top of the leaflet engaging portion so that there is good contact between the material covering and the leaflets/annulus to promote sealing, healing, and possibly tissue in-growth.

Third, portions of the lower radial struts 162D (e.g., those closes to the outflow end of the framework 152, such as 162H) may be positioned within the annulus of the native valve. Since portions of the lower radial struts 162D may curve radially outward, this shape may help further seal the framework 152 with the native annulus, further limiting the passage of blood around the framework/valve.

The framework 152 of the present example may be composed of a single unitary body, such as laser cut from a shape memory tube (e.g., Nitinol tube). Alternatively, one or more of the struts of the framework may be welded or otherwise attached to each other. Alternatively, some of the components may be separate from each other, only connected by other materials, such as the material covering 154 or other attachment mechanisms. For example, the body portion 160, the leaflet engagement portion 158, and/or the atrial flange portion 156 may not be directly attached to each other in any combination. If shape memory material is used for the framework 152, the framework may be cut to a desired pattern and then heat set to impart a desired shape in its expanded configuration.

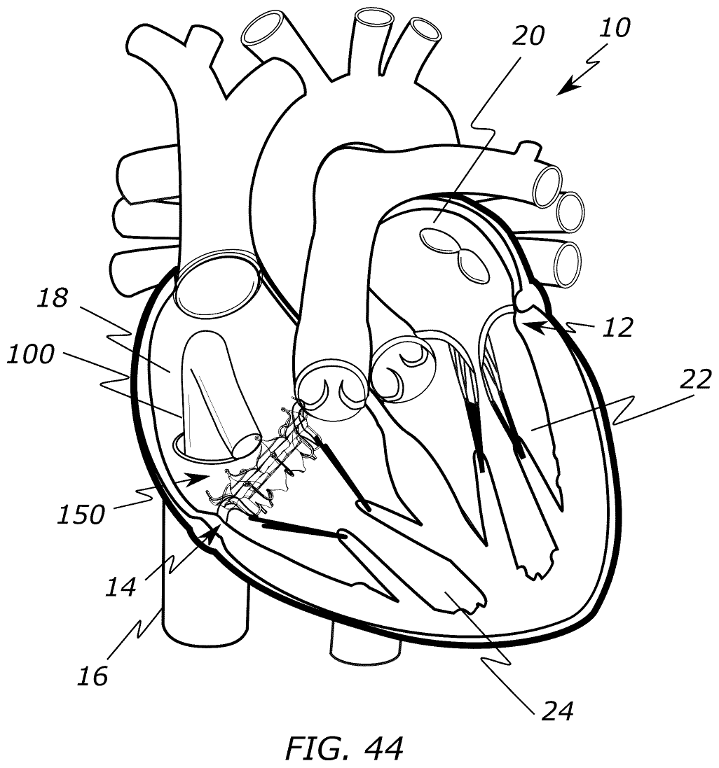

The artificial heart valve 150 may deploy from a delivery device 100 or delivery device 100′. In that respect, the engagement struts 164 may begin in a compressed configuration with their ends (rounded enlargement 164A) positioned distally away from the body portion 160 within the delivery device 100 or delivery device 100′. As the artificial heart valve 150 is pushed out of the first tubular member 108 or the first tubular member 108 is retracted from over the artificial heart valve 150, the engagement struts 164 extend radially outward from the delivery device 100 or delivery device 100′, and then, as the artificial heart valve 150 continues to advance or be exposed, the engagement struts 164 bend backward such that the rounded enlarged end 164A is positioned in an inflow direction relative to the outflow end of the body portion 160. In other words, as deployment occurs, the engagement struts 164 bend radially backward or invert which allows them to capture the native valve leaflets 14B with the body portion 160 and press against the valve annulus 14A, as seen in FIG. 44.

In a compressed configuration within the delivery device 100 or delivery device 100′, it should be noted that the prosthesis engagement portions 162A with an aperture may be located at a proximal end of the compressed artificial heart valve 150 while engagement struts 164 are constrained distally such that the rounded portion 164A is at a distal most location within the delivery device 100 or delivery device 100′. In that respect, apertures are located at both the proximal and distal ends of the artificial heart valve 150 in its compressed configuration. Additionally, aperture portion 166C also includes an aperture midway along the length of the compressed configuration. These apertures may be engaged with features of the delivery device 100 or delivery device 100′, such as posts, hooks, tethers, or similar structures that help retain portions of the support structure until 150 until fully deployed.

As previously discussed, although the artificial heart valve 150 is mostly described in this specification, it is specifically contemplated that a valve mechanism 170, such as prosthetic or biological valve leaflets, be attached within the valve support mechanism, as seen in FIGS. 42 and 43.

FIG. 44 illustrates one approach to delivering a support structure 100 within a tricuspid valve 14 of a heart 10 by advancing a delivery catheter through the inferior vena cava 16 and into the right atrium 18, such that the support structure is delivered from an inflow or atrial end relative to the tricuspid valve 14.

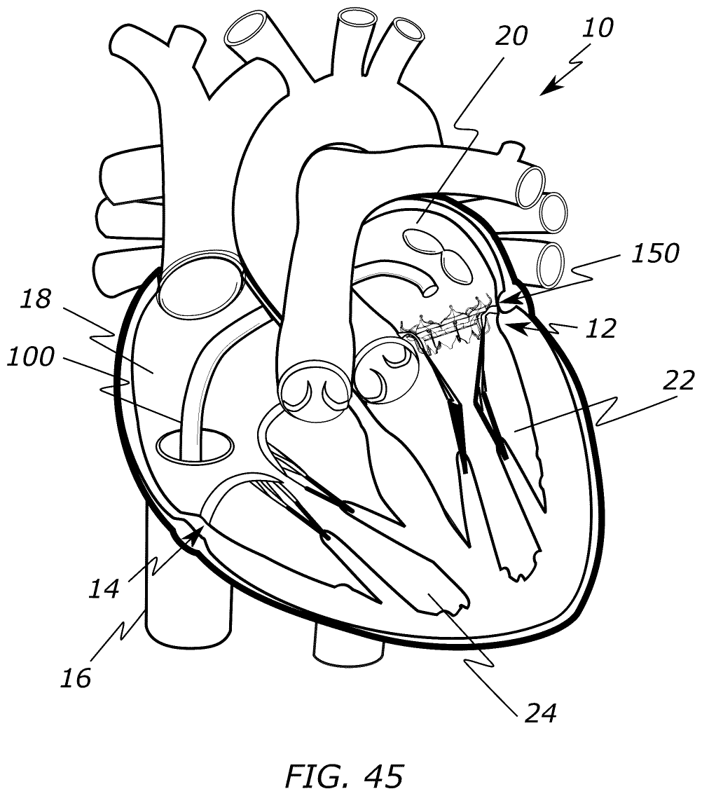

FIG. 45 illustrates another approach to delivering a support structure 100 with a mitral valve 12 by performing a transeptal procedure to allow the delivery catheter to pass through the septum between the right atrium 18 and the left atrium 20. This allows the support structure to be delivered from an inflow or atrial end relative to the mitral valve 12.

1. A delivery system for a heart prosthesis, comprising:
a tubular loading member comprising a notch passing entirely through a wall of the tubular loading member and located at a distal edge of the tubular loading member; and,
a prosthesis retention member positioned within the tubular loading member; the prosthesis retention member comprising a plurality of prosthesis retention areas shaped to engage prosthesis engagement portions of a heart prosthesis;
wherein the tubular loading member and the prosthesis retention member are longitudinally and rotationally movable relative to each other; and, wherein the notch includes a plurality of rotational positions that completely radially expose one of the plurality of prosthesis retention areas while covering a remaining portion of the plurality of prosthesis retention areas;
wherein the tubular loading member comprises a prosthesis loading tool having a tubular structure; wherein the tubular loading member is removable from the delivery system;
further comprising a notch cover member that is removably positioned over the notch of the tubular loading member and at least partially around the tubular loading member;
wherein the notch cover member further comprises a ridge positioned on an inner surface of the notch cover member and that is sized to fit within the notch of the prosthesis loading tool.

2. The delivery system of claim 1, wherein the plurality of prosthesis retention areas are positioned circumferentially around a body of the prosthesis retention member.

3. The delivery system of claim 2, wherein the notch is about a same size or larger than each of the plurality of prosthesis retention areas.

4. The delivery system of claim 3, wherein the notch has a width and shape sized to allow the prosthesis engagement portions pass through the notch.

5. (canceled)

6. The delivery system of claim 1, wherein the tubular loading member comprises a distal region having a reduced diameter relative to a proximal region.

7. The delivery system of claim 1, further comprising a funnel member removably connected to the tubular loading member.

8-9. (canceled)

10. The delivery system of claim 1, wherein the notch cover member is an open tubular shape.

11. The delivery system of claim 1, wherein the prosthesis engagement portions are enlarged regions on a proximal portion of the heart prosthesis.

12. The delivery system of claim 11, wherein the enlarged regions are eyelets.

13. The delivery system of claim 2, wherein the plurality of prosthesis retention areas comprise depressions having rectangular shape adjacent to a circular shape.

14. The delivery system of claim 2, wherein the plurality of prosthesis retention areas comprise depressions, grooves, posts, walls, or any combination of the same.

15. The delivery system of claim 1 wherein the tubular loading member comprises a first tubular member and the notch is located at a distal edge of the first tubular member; wherein the first tubular member is non-removably fixed to the delivery system.

16. The delivery system of claim 1, further comprising a first tubular member longitudinally movable relative to the prosthesis retention member.

17. The delivery system of claim 16, further comprising a second tubular member positioned within a lumen of the first tubular member and connected to the prosthesis retention member.

18. The delivery system of claim 17, further comprising a shaft positioned within a lumen of the second tubular member; wherein a distal region of the shaft comprises a nosecone.

19. The delivery system of claim 18, further comprising a third tubular member disposed around the first tubular member, wherein the third tubular member forms an outer tube of the delivery system.

20. The delivery system of claim 19, further comprising a handle assembly, wherein the handle assembly comprises a movement mechanism connected to the first tubular member that moves the first tubular member distally and proximally relative to the handle assembly.

21. The delivery system of claim 20, wherein the movement mechanism comprises an outer tube that rotates relative to an outer handle housing; and wherein an inner surface of the outer tube is engaged with a nut via threaded surfaces that cause the nut to longitudinally move when the outer tube is rotated; and wherein the nut is connected to the first tubular member.

22. The delivery system of claim 21, wherein the first outer tube comprises a distal tubular portion comprised of a rigid material and comprises a proximal tubular portion comprised of a flexible material.

23. The delivery system of claim 22, wherein the heart prosthesis is a heart valve comprising an atrial flange portion, a leaflet engaging portion, and a body portion.

24. The delivery system of claim 23, wherein the leaflet engaging portion comprise a plurality of engagement struts that are positioned proximally of the body portion when the heart valve is in a radially compressed configuration and positioned alongside the body portion when the heart valve is in a radially expand configuration.

25. A method of loading a heart prosthesis on to a delivery system, comprising:
positioning a proximal end of the heart prosthesis near a prosthesis retention member of a delivery device;
rotating a tubular loading member at a distal region of the delivery device so that a notch on a distal end of the tubular loading member aligns with a first prosthesis retention area of plurality of prosthesis retention areas;
placing a first prosthesis engagement portion of a heart prosthesis through the notch and into first prosthesis retention area of the plurality of prosthesis retention areas;
rotating the tubular loading member so that the notch aligns with a second prosthesis retention area of the plurality of prosthesis retention areas;
placing a second enlarged retention portion of the heart prosthesis through the notch and into the second prosthesis retention area of the plurality of prosthesis retention areas;
further rotating the tubular loading member, aligning the notch with further retention area of the plurality of prosthesis retention areas, and placing further enlarged retention portions of the heart prosthesis into corresponding retention areas of the plurality of prosthesis retention areas; and,
covering the notch to prevent the heart prosthesis from radially expanding;
wherein the tubular loading member is a prosthesis loading tool that is removable from the delivery device or a first tubular member that is non-removably fixed to the delivery device
wherein covering the heart prosthesis comprises placing notch cover member 134 over the notch
wherein placing a spacer member over the notch further comprises placing a ridge on an inner surface of the spacer into the notch.

26-28. (canceled)

29. The method of claim 25, wherein covering the heart prosthesis comprises retracting the prosthesis retention member and the heart prosthesis into first tubular member of the delivery device.

30. The method of claim 29, further comprising removing the prosthesis loading tool and the notch cover member.

31. The method of claim 30, positioning the proximal end of the heart prosthesis through a funnel member located at a distal end of the prosthesis loading tool.

32. The method of claim 31, wherein retracting the prosthesis retention member and the heart prosthesis into the first tubular member of the delivery device further comprises rotating a first portion of a handle connected to a proximal end of the first tubular member.
